SEQUENCE LISTING (37 sequences)

SEQ ID NO: 1 - protein, 533 aa, Chlamydia trachomatis
MSNSFRDQEQGLQAVFRAARVISHMFSQTIGPYGFSTIVHNVQDTRTTQDSQSMLKDILFPDVFENIGMKLIRDTALRTRMRFGDGAKTTALLIEALLAEGMTGIQKGLDPHEIHRGMLLAEKKIQEVFYRETFPLSDLEHTVYVSSIARRCNSEIASVLSSAVGYGGKNGYYIVEEHEEHETYWHAEEHAVWDFGYASPYFITHAETGTVEYSQVYILVSEQPLHYSNPSFLTFLQSVVQAGKTPLVILAEAFDKELLAMLEMNQIERVFPVCAVKVSGKHARESLEDIAVLTGATLLSEMDFEDSEEERITNRLGFVAGICVSSTSLCVPRETDNKQRMAEHCAFLQDKLSFSQEEEASARLRRRLARLSSGEVCIHIAADCIPQEEIGYITSSIRAMTESLRSGCLPGGGCAFIRAAREISVPLALSPSERFGFLAVLSAAEKPFRAIVTRSRRVEEEVFSEVFSQADWRVGFNGVSGFVEDIVSQGICDGASCIQYALSHAVGTTGLLLTSALFIASQEPMLREENSEE

SEQ ID NO: 2 - protein, 544 aa, Chlamydia trachomatis
MVAKNIKYNEEARKKIQKGVKTLAEAVKVTLGPKGRHVVIDKSFGSPQVTKDGVTVAKEVELADKHENMGAQMVKEVASKTADKAGDGTTTATVLAEAIYTEGLRNVTAGANPMDLKRGIDKAVKVVVDQIRKISKPVQHHKEIAQVATISANNDAEIGNLIAEAMEKVGKNGSITVEEAKGFETVLDIVEGMNFNRGYLSSYFATNPETQECVLEDALVLIYDKKISGIKDFLPVLQQVAESGRPLLIIAEDIEGEALATLVVNRIRGGFRVCAVKAPGFGDRRKAMLEDIAILTGGQLISEELGMKLENANLAMLGKAKKVIVSKEDTTIVEGMGEKEALEARCESIKKQIEDSSSDYDKEKLQERLAKLSGGVAVIRVGAATEIEMKEKKDRVDDAQHATIAAVEEGILPGGGTALIRCIPTLEAFLPMLTNEDEQIGARIVLKALSAPLKQIAANAGKEGAIIFQQVMSRSANEGYDALRDAYTDMLEAGILDPAKVTRSALESAASVAGLLLTTEALIAEIPEEKPAAAPAMPGAGMDY

SEQ ID NO: 3 - protein, 872 aa, Chlamydia trachomatis
MPHDNNEMHRNTIHQLFTGLDKAYQIVKGFYGPAYSSSSKDFFKGRGYHILSRIELSDPFERIGVYFARSLAKRIHKRHADGVISSVILLRAFLKASIPFIDQGLSPRLLASALASQKEAVCAYLHSHSFLLKDASKVLGLIRSHLPDPLIGEAFAEAVAYTGHEGAVALSQRSGSTLHLVKGIQTQKGYRVPSFFPHDSFHENPIVAPKIFVTDQKIHCLFPFLPLLKKFSEEQTPLIIFCKEIAPDPLATCIANRIAGLLDVLVVTIPDTTLLEDIALLTGTTVFSSPPFSNKPPIELPLLGSCTWAELSRDHTLLVCENLVPEVVKLKVRQLDHAIHNAEDETSRKLLKKRKHRLENSIAIIPVKQDTTPLHELALKTLNSTQESGFVLGGGAALLYATQSLSSSPEHSQEEQAAVQILQTACRTLLEQLVNSVYMDGKLVADKLCSLGTPSLGFNVVSQQIEDMISAGIITPLNVVLDIFSCSLHTAVDLLLASFTTPPTPAAKEKKTMVSFDLNDPVRNTDNHYRNINRMLNSATCAAGGIGLLTPVVCSPMGAFCFAQGPSSAEDLGHRIQHFVACSGPAAGFYSLSNERIMFEEAAVPSVLEAVEATFWISAFARLRGNEPSTCDTVMMSCVIGCISLVCGAMFVAIVSCAVKISRIVRTMTQAHALRETIQRQLAARATDMRSAYSKLKGIIAIRALNEVERGHRKLRNKMITAFVANALITLAFCALLASAVIAAFFFGAASAGLASVFFGCLWGGIGALAVGVLVGIVSGICQRNYKVEAARCIQRGALYALVLEKMQRFPKEFLKDGVAKSVVAIQAGESLDTGELAWEEMPSITACLGREGMDAQAYSFLSASPLDARIE

SEQ ID NO: 4 - protein, 360 aa, Chlamydia trachomatis
MVSFDLNDPVRNTDNHYRNINRMLNSATCAAGGIGLLTPVVCSPMGAFCFAQGPSSAEDLGHRIQHFVACSGPAAGFYSLSNERIMFEEAAVPSVLEAVEATFWISAFARLRGNEPSTCDTVMMSCVIGCISLVCGAMFVAIVSCAVKISRIVRTMTQAHALRETIQRQLAARATDMRSAYSKLKGIIAIRALNEVERGHRKLRNKMITAFVANALITLAFCALLASAVIAAFFFGAASAGLASVFFGCLWGGIGALAVGVLVGIVSGICQRNYKVEAARCIQRGALYALVLEKMQRFPKEFLKDGVAKSVVAIQAGESLDTGELAWEEMPSITACLGREGMDAQAYSFLSASPLDARIE

SEQ ID NO: 5 - protein, 639 aa, Chlamydia trachomatis
MNVRTYSVQRGGVKTISASAVPPTAAVLSRKKRAIEEKKEEASSGKIENLDASKYDLTPKNIEEKLGITPEQKSTVKDLLNKLKKVISAYNSMPDKNSEAGQNSLIQQGKYVDAIQKKLPASSQAQPKQAKAKEQKAEEKPKTTPIEGVLETIKTEFKGHRVPVEKIIHGIWIAGAPPDGIEDYMRVFLDTYEGFDFYFWVDENAYAAAKFSSILKKVAFDAAIQDLRSATDESTKAFVKDYDELKQKYEKKVAETTSQAEKDQYLKDLKDLLEKFTKISDEIRGKFDRLFLKNVIVAQNGFFNFCLLKGLGNINDETRAEYLEKELKLPTEEIEQYKKLKETNKEKIAAIVKQLNEKLGSDRVKIKDIKELQSMKQARNVYNYEQEMFLRWNYAAATDQIRMYMLEELGGLYTDLDMMPSYSQEVLELIKKHSDGNRMFEDMSSRRAISDAVLKMAVGKATTVSMEEVAKDIDVSRLTEEDKTKLNALFKDLEPFAKPDSKGAEAEGGEGAKGMKKSFFQPIDLNIVRNTMPILRRYHHYPELGWFIRGLNGLMVSHKGSTAVSAVIVGQQAAYQELAALRQDVLSGEFFHSLENLTHRNHKERIGNHLVANYLAKSLFFDYCQDSVMPEAVSTLGIR

SEQ ID NO: 6 - protein, 159 aa, Chlamydia trachomatis
MSAATSQIGDTQYVSSLPPLEPLGTPPIAELLFSIYSLLLEAVEIRQETILTQSKQLNDNTNIQQQLNQETNQIKYAVVGSGAKEDEITRVQNQNQNYSAQRSNIQDQLVTARQNGQIILSHASTNINIMQQIAQQNSSFIKTLNSVGSTVNQLNKPLS

SEQ ID NO: 7 - protein, 135 aa, Chlamydia trachomatis
MFIKSFVHLCLLFSLTPTRLFSAAPISAHPSVTKKLSLRQLFKRFLPETSHNKKERYYHFLKHVSHALKRPEIWNDLQNLLLILMQFEQFANQEVGSRRYEEILSQIIEKRIHFLLSEQQVSELTDILAPKPGDL

SEQ ID NO: 8 - protein, 83 aa, Chlamydia trachomatis
MASGSCSAFNFNQMLDGVCKYVQGVQQYLTELETSTQGTVDLGTMFNLQFRMQILSQYMESVSNILTAVNTEMITMARAVKGS

SEQ ID NO: 9 - protein, 90 aa, Chlamydia trachomatis
MKICREICDAILKRDFATFEIERTYKNGSIVYTSRQCPGVEIREIGDQAKAILHNQEELRQAVATLGLKHLIIPPLSIYKHSLLYKRLVI

SEQ ID NO: 10 - protein, 119 aa, Chlamydia trachomatis
MDSFCLNLLKVIVKAIDNKKGRNPVVLDVQNISQLTDYFVFVEGNVGVHIKAIADTIIEELKKLKVYPLNVEGLSHSDWVVIDYGFIVIHLFVSSVREQYCLEELWKDGAIITSDCLAS

SEQ ID NO: 11 - protein, 56 aa, Chlamydia trachomatis
MLRLFCLGYFPQLFEKQKNLLEDLIYNKLFVEKTVVMGEGACVVNLAIGGGETILL

SEQ ID NO: 12 - protein, 82 aa, Chlamydia trachomatis
MGQDHRRKFLKKVSFVKKQAAFAGNFIEEIKKIEWVNKRDLKRYVKIVLMNIFGFGFSIYCVDLALRKSLSLFGKVTSFFFG

SEQ ID NO: 13 - protein, 1005 aa, Chlamydia trachomatis
MTNSISGYQPTVTTSTSSTTSASGASGSLGASSVSTTANATVTQTANATNSAATSSIQTTGETVVNYTNSASAPNVTVSTSSSSTQATATSNKTSQAVAGKITSPDTSESSETSSTSSSDHIPSDYDDVGSNSGDISNNYDDVGSNNGDISSNYDDAAADYEPIRTTENIYESIGGSRTSGPENTSGGAAAALNSLRGSSYSNYDDAAADYEPIRTTENIYESIGGSRTSGPENTSGGAAAALNSLRGSSYSNYDDAAADYEPIRTTENIYESIGGSRTSGPENTSDGAAAAALNSLRGSSYTTGPRNEGVFGPGPEGLPDMSLPSYDPTNKTSLLTFLSNPHVKSKMLENSGHFVFIDTDRSSFILVPNGNWDQVCSIKVQNGKTKEDLDIKDLENMCAKFCTGFSKFSGDWDSLVEPMVSAKAGVASGGNLPNTVIINNKFKTCVAYGPWNSQEASSGYTPSAWRRGHRVDFGGIFEKANDFNKINWGTQAGPSSEDDGISFSNETPGAGPAAAPSPTPSSIPIINVNVNVGGTNVNIGDTNVNTTNTTPTTQSTDASTDTSDIDDINTNNQTDDINTTDKDSDGAGGVNGDISETESSSGDDSGSVSSSESDKNASVGNDGPAMKDILSAVRKHLDVVYPGENGGSTEGPLPANQTLGDVISDVENKGSAQDTKLSGNTGAGDDDPTTTAAVGNGAEEITLSDTDSGIGDDVSDTASSSGDESGGVSSPSSESNKNTAVGNDGPSGLDILAAVRKHLDKVYPGDNGGSTEGPLQANQTLGDIVQDMETTGTSQETVVSPWKGSTSSTESAGGSGSVQTLLPSPPPTPSTTTLRTGTGATTTSLMMGGPIKADIITTGGGGRIPGGGTLEKLLPRIRAHLDISFDAQGDLVSTEEPQLGSIVNKFRQETGSRGILAFVESAPGKPGSAQVLTGTGGDKGNLFQAAAAVTQALGNVAGKVNLAIQGQKLSSLVNDDGKGSVGRDLFQAAAQTTQVLSALIDTVG

SEQ ID NO: 14 - protein, 591 aa, Chlamydia trachomatis
MSIRGVGGNGNSRIPSHNGDGSNRRSQNTKGNNKVEDRVCSLYSSRSNENRESPYAVVDVSSMIESTPTSGETTRASRGVFSRFQRGLVRVADKVRRAVQCAWSSVSTRRSSATRAAESGSSSRTARGASSGYREYSPSAARGLRLMFTDFWRTRVLRQTSPMAGVFGNLDVNEARLMAAYTSECADHLEANKLAGPDGVAAAREIAKRWEQRVRDLQDKGAARKLLNDPLGRRTPNYQSKNPGEYTVGNSMFYDGPQVANLQNVDTGFWLDMSNLSDVVLSREIQTGLRARATLEESMPMLENLEERFRRLQETCDAARTEIEESGWTRESASRMEGDEAQGPSRAQQAFQSFVNECNSIEFSFGSFGEHVRVLCARVSRGLAAAGEAIRRCFSCCKGSTHRYAPRDDLSPEGASLAETLARFADDMGIERGADGTYDIPLVDDWRRGVPSIEGEGSDSIYEIMMPIYEVMDMDLETRRSFAVQQGHYQDPRASDYDLPRASDYDLPRSPYPTPPLPPRYQLQNMDVEAGFREAVYASFVAGMYNYVVTQPQERIPNSQQVEGILRDMLTNGSQTFRDLMRRWNREVDRE

SEQ ID NO: 15 - protein, 78 aa, Chlamydia trachomatis
MKEFLAYIVKNLVDKPEEVHLKEVQGTNTIIYELTVAKGDIGKIIGKEGRTIKAIRTLLVSVASRDNVKVSLEIMEER

SEQ ID NO: 16 - protein, 62 aa, Chlamydia trachomatis
MPDPCSNNSKDTPYCRIHSDEENTTESSVQSSSPEDGVSVSSASMQIQATAEHVSLMVIGGG

SEQ ID NO: 17 - protein, 215 aa, Chlamydia trachomatis
MSCSNINSGAPATLFAKGAAAISSRVDTMRQMCSARVGAKTCGIALTIIGLLVATAGVVIAAVGIGTPASLAAGMILVMVGSLLLGLGLARARSRRVEVERHLEVVSMQIEGLKEEYKALFEVYQVNLEARRAVSKHCQDLEEQILDLCKRYAETVCSIEEDAEQEIRHQTEGFKQRLQQSQNTCSQLTAELCKLRSENKTLSERLQVQASRRKK

SEQ ID NO: 18 - protein, 105 aa, Chlamydia trachomatis
MEIRYFLARPLLEEEVCRLANNRKNFLFDAEKYLIPICYKQTIYLAKPLSRFPMALEVWELHVQHVISLLKQQFGILTDHAPILLACEARQVVLLESLDSFVNIS

SEQ ID NO: 19 - protein, 280 aa, Chlamydia trachomatis
MKKPVFTGGAPIPGISTEEGTGVKDQNLWMRNATLKVEGDATIDDTLTSRDLKVTGPTIHTDLDLSVGGDVKGGRTVLGETVLEGDFNIKCNQGQVPQFTNLSDPLSARDAITFDYYRDRSTQAYNCATHRNGALVNGNRFIDLRLHNSEDSESYTPMYRNRFYWKDNDQKKLYLKSPGIYQVAFQIFRSGGYHSGNDDPTIFLRLYTSAYEYTNLCTGDTRGFNPGNTTNTSLYSIFSIPSIGNEHPFIQVFTKIHVNIAYSMINVIWFPFGSSYKEAD

SEQ ID NO: 20 - protein, 248 aa, Chlamydia trachomatis
MASEYEHFGNLSPEDHVKEVQDLHKVCKGEPHQTKKGYWYHLTCDAIDCGVFLFFIRTIFFLVPAIPVTSYGKILFATGISWIFYTSCKRAQAAWAYIELTHRNMLQEKKEIETNPEQERIELAVLYANQGFQEPLISQMLDFVCSDSSLLLSTMLREELHIQLEDYPHPLKQGNVKALGGILGLLLFAPITLAVSYTIAAILASFMIGVLFAVKTRLIKNAIAPAIVWGVGMFITAISLCCSLIRLF

SEQ ID NO: 21 - protein, 520 aa, Chlamydia trachomatis
MNKKERINKKNASTKFQRSTPTRALLSIGSQQLSSFTKLSFDGQAKLTGVATPTRDTDVVPLQYLQARYLSKNDPNPGYLPIHGGNMTGNINMGTHSVFNLKQPEKPKIELPSETDKPKDPREEDGFAEKTAEEQEQEIKEYNTKLAEYQKKIDDYNAAWEAFYSEAATVKYVKGIVDKILNNDKLSTALNSATEVEKKIALAQKALGIEITINPDADTNPDTDQETPDPAPVADTEEKESPPLSYNDLPSVIKNSQFVVTQSQNKITGDLKMTNAQIANIKTPDTGDSNYAANVTYLESKLKQPQRAFLSNTLPTESSSSISLNGHIPWLSTTNGSSSPAEPDFKSKLADQCFDTSSQENLKVKTAGLLVLSVRGTWSPTTSPITNGSTPTPTTISVNLTVTPDNSSRTNTSSSGSDSSGDASATTLTIPLTLYSGESVQLQLPITTTSSVKIATTTSQTSNGGSDTSSQITLSSWSWEAALYPTDVTVTNKTTPPTTETPSSPSPSSPNSESTEGQTP

SEQ ID NO: 22 - protein, 239 aa, Chlamydia trachomatis
MSYSFFRRKLAGIEFIECPGDPEAPVIIFCHGYGASADHLTFFPTMCVCANLRPTWVFPHGIEQLPYQLGGGRAWFPLDTVLFEKLISSQEITPDTDRLYQQLLDVDFEKPKQALEGLIHELERDRSEVIIGGFSQGAMMTTHLMLSSRLPYRGALICSGAAVPNQSWEENASLCGKTPYIQSHGYDDPILPYFLGERLYKVLTASLKGEMVSFHGGHEIPVVMMQKIQESIALWSQLT

SEQ ID NO: 23 - protein, 975 aa, Chlamydia trachomatis
MNRVIEIHAHYDQRQLSQSPNTNFLVHHPYLTLIPKFLLGALIVYAPYSFAEMELAISGHKQGKDRDTFTMISSCPEGTNYIINRKLILSDFSLLNKVSSGGAFRNLAGKISFLGKNSSASIHFKHININGFGAGVFSESSIEFTDLRKLVAFGSESTGGIFTAKEDISFKNNHHIAFRNNITKGNGGVIQLQGDMKGSVSFVDQRGAIIFTNNQAVTSSSMKHSGRGGAISGDFAGSRILFLNNQQITFEGNSAVHGGAIYNKNGLVEFLGNAGPLAFKENTTIANGGAIYTSNFKANQQTSPILFSQNHANKKGGAIYAQYVNLEQNQDTIRFEKNTAKEGGGAITSSQCSITAHNTIIFSDNAAGDLGGGAILLEGKKPSLTLIAHSGNIAFSGNTMLHITKKASLDRHNSILIKEAPYKIQLAANKNHSIHFFDPVMALSASSSPIQINAPEYETPFFSPKGMIVFSGANLLDDAREDVANRTSIFNQPVHLYNGTLSIENGAHLIVQSFKQTGGRISLSPGSSLALYTMNSFFHGNISSKEPLEINGLSFGVDISPSNLQAEIRAGNAPLRLSGSPSIHDPEGLFYENRDTAASPYQMEILLTSDKIVDISKFTTDSLVTNKQSGFQGAWHFSWQPNTINNTKQKILRASWLPTGEYVLESNRVGRAVPNSLWSTFLLLQTASHNLGDHLCNNRSLIPTSYFGVLIGGTGAEMSTHSSEEESFISRLGATGTSIIRLTPSLTLSGGGSHMFGDSFVADLPEHITSEGIVQNVGLTHVWGPLTVNSTLCAALDHNAMVRICSKKDHTYGKWDTFGMRGTLGASYTFLEYDQTMRVFSFANIEATNILQRAFTETGYNPRSFSKTKLLNIAIPIGIGYEFCLGNSSFALLGKGSIGYSRDIKRENPSTLAHLAMNDFAWTTNGCSVPTSAHTLANQLILRYKACSLYITAYTINREGKNLSNSLSCGGYVGF

SEQ ID NO: 24 - protein, 1751 aa, Chlamydia trachomatis
MKWLSATAVFAAVLPSVSGFCFPEPKELNFSRVGTSSSTTFTETVGEAGAEYIVSGNASFTKFTNIPTTDTTTPTNSNSSSSNGETASVSEDSDSTTTTPDPKGGGAFYNAHSGVLSFMTRSGTEGSLTLSEIKITGEGGAIFSQGELLFTDLTGLTIQNNLSQLSGGAIFGESTISLSGITKATFSSNSAEVPAPVKKPTEPKAQTASETSGSSSSSGNDSVSSPSSSRAEPAAANLQSHFICATATPAAQTDTETSTPSHKPGSGGAIYAKGDLTIADSQEVLFSINKATKDGGAIFAEKDVSFENITSLKVQTNGAEEKGGAIYAKGDLSIQSSKQSLFNSNYSKQGGGALYVEGDINFQDLEEIRIKYNKAGTFETKKITLPKAQASAGNADAWASSSPQSGSGATTVSNSGDSSSGSDSDTSETVPATAKGGGLYTDKNLSITNITGIIEIANNKATDVGGGAYVKGTLTCENSHRLQFLKNSSDKQGGGIYGEDNITLSNLTGKTLFQENTAKEEGGGLFIKGTDKALTMTGLDSFCLINNTSEKHGGGAFVTKEISQTYTSDVETIPGITPVHGETVITGNKSTGGNGGGVCTKRLALSNLQSISISGNSAAENGGGAHTCPDSFPTADTAEQPAAASAATSTPESAPVVSTALSTPSSSTVSSLTLLAASSQASPATSNKETQDPNADTDLLIDYVVDTTISKNTAKKGGGIYAKKAKMSRIDQLNISENSATEIGGGICCKESLELDALVSLSVTENLVGKEGGGLHAKTVNISNLKSGFSFSNNKANSSSTGVATTASAPAAAAASLQAAAAAVPSSPATPTYSGVVGGAIYGEKVTFSQCSGTCQFSGNQAIDNNPSQSSLNVQGGAIYAKTSLSIGSSDAGTSYIFSGNSVSTGKSQTTGQIAGGAIYSPTVTLNCPATFSNNTASMATPKTSSEDGSSGNSIKDTIGGAIAGTAITLSGVSRFSGNTADLGAAIGTLANANTPSATSGSQNSITEKITLENGSFIFERNQANKRGAIYSPSVSIKGNNITFNQNTSTHDGSAIYFTKDATIESLGSVLFTGNNVTATQASSATSGQNTNTANYGAAIFGDPGTTQSSQTDAILTLLASSGNITFSNNSLQNNQGDTPASKFCSIAGYVKLSLQAAKGKTISFFDCVHTSTKKIGSTQNVYETLDINKEENSNPYTGTIVFSSELHENKSYIPQNAILHNGTLVLKEKTELHVVSFEQKEGSKLIMKPGAVLSNQNIANGALVINGLTIDLSSMGTPQAGEIFSPPELRIVATTSSASGGSGVSSSIPTNPKRISAAAPSGSAATTPTMSENKVFLTGDLTLIDPNGNFYQNPMLGSDLDVPLIKLPTNTSDVQVYDLTLSGDLFPQKGYMGTWTLDSNPQTGKLQARWTFDTYRRWVYIPRDNHFYANSILGSQNSMIVVKQGLINNMLNNARFDDIAYNNFWVSGVGTFLAQQGTPLSEEFSYYSRGTSVAIDAKPRQDFILGAAFSKMVGKTKAIKKMHNYFHKGSEYSYQASVYGGKFLYFLLNKQHGWALPFLIQGVVSYGHIKHDTTTLYPSIHERNKGDWEDLGWLADLRISMDLKEPSKDSSKRITVYGELEYSSIRQKQFTEIDYDPRHFDDCAYRNLSLPVGCAVEGAIMNCNILMYNKLALAYMPSIYRNNPVCKYRVLSSNEAGQVICGVPTRTSARAEYSTQLYLGPFWTLYGNYTIDVGMYTLSQMTSCGARMIF

SEQ ID NO: 25 - protein, 1770 aa, Chlamydia trachomatis
MKFMSATAVFAAALSSVTEASSIQDQIKNTDCNVSKLGYSTSQAFTDMMLADNTEYRAADSVSFYDFSTSSRLPRKHLSSSSEASPTTEGVSSSSSGETDEKTEEELDNGGIIYAREKLTISESQDSLSNQSIELHDNSIFFGEGEVIFDHRVALKNGGAIYGEKEVVFENIKSLLVEVNIAVEKGGSVYAKERVSLENVTEATFSSNGGEQGGGGIYSEQDMLISDCNNVHFQGNAAGATAVKQCLDEEMIVLLAECVDSLSEDTLDSTPETEQTESNGNQDGSSETEDTQVSESPESTPSPDDVLGKGGGIYTEKSLTITGITGTIDFVSNIATDSGAGVFTKENLSCTNTNSLQFLKNSAGQHGGGAYVTQTMSVTNTTSESITTPPLIGEVIFSENTAKGHGGGICTNKLSLSNLKTVTLTKNSAKESGGAIFTDLASIPITDTPESSTPSSSSPASTPEVVASAKINRFFASTAKPAAPSLTEAESDQTDQTETSDTNSDIDVSIENILNVAINQNTSAKKGGAIYGKKAKLSRINNLELSGNSSQDVGGGLCLTESVEFDAIGSLLSHYNSAAKEGGAIHSKTVTLSNLKSTFTFADNTVKAIVESTPEAPEEIPPVEGEESTATEDPNSNTEGSSANTNLEGSQGDTADTGTGDVNNESQDTSDTGNAESEEQLQDSTQSNEENTLPNSNIDQSNENTDESSDSHTEEITDESVSSSSESGSSTPQDGGAASSGAPSGDQSISANACLAKSYAASTDSSPVSNSSGSEEPVTSSSDSDVTASSDNPDSSSSGDSAGDSEEPTEPEAGSTTETLTLIGGGAIYGETVKIENFSGQGIFSGNKAIDNTTEGSSSKSDVLGGAVYAKTLFNLDSGSSRRTVTFSGNTVSSQSTTGQVAGGAIYSPTVTIATPVVFSKNSATNNANNTTDTQRKDTFGGAIGATSAVSLSGGAHFLENVADLGSAIGLVPGTQNTETVKLESGSYYFEKNKALKRATIYAPVVSIKAYTATFNQNRSLEEGSAIYFTKEASIESLGSVLFTGNLVTLTLSTTTEGTPATTSGDVTKYGAAIFGQIASSNGSQTDNLPLKLIASGGNICFRNNEYRPTSSDTGTSTFCSIAGDVKLTMQAAKGKTISFFDAIRTSTKKTGTQATAYDTLDINKSEDSETVNSAFTGTILFSSELHENKSYIPQNVVLHSGSLVLKPNTELHVISFEQKEGSSLVMTPGSVLSNQTVADGALVINNMTIDLSSVEKNGIAEGNIFTPPELRIIDTTTGGSGGTPSTDSESNQNSDDTEEQNNNDASNQGESANGSSSPAVAAAHTSRTRNFAAAATATPTTTPTATTTTSNQVILGGEIKLIDPNGTFFQNPALRSDQQISLLVLPTDSSKMQAQKIVLTGDIAPQKGYTGTLTLDPDQLQNGTISVLWKFDSYRQWAYVPRDNHFYANSILGSQMLMVTVKQGLLNDKMNLARFEEVSYNNLWISGLGTMLSQVGTPTSEEFTYYSRGASVALDAKPAHDVIVGAAFSKMIGKTKSLKRENNYTHKGSEYSYQASVYGGKPFHFVINKKTEKSLPLLLQGVISYGYIKHDTVTHYPTIRERNKGEWEDLGWLTALRVSSVLRTPAQGDTKRITVYGELEYSSIRQKQFTETEYDPRYFDNCTYRNLAIPMGLAFEGELSGNDILMYNRFSVAYMLSIYRNSPTCKYQVLSSGEGGEIICGVPTRNSARGEYSTQLYLGPLWTLYGSYTIEADAHTLAHMMNCGARMTF

SEQ ID NO: 26 - protein, 1531 aa, Chlamydia trachomatis
MSSEKDIKSTCSKFSLSVVAAILASVSGLASCVDLHAGGQSVNELVYVGPQAVLLLDQIRDLFVGSKDSQAEGQYRLIVGDPSSFQEKDADTLPGKVEQSTLFSVTNPVVFQGVDQQDQVSSQGLICSFTSSNLDSPRDGESFLGIAFVGDSSKAGITLTDVKASLSGAALYSTEDLIFEKIKGGLEFASCSSLEQGGACAAQSILIHDCQGLQVKHCTTAVNAEGSSANDHLGFGGGAFFVTGSLSGEKSLYMPAGDMVVANCDGAISFEGNSANFANGGAIAASGKVLFVANDKKTSFIENRALSGGAIAASSDIAFQNCAELVFKGNCAIGTEDKGSLGGGAISSLGTVLLQGNHGITCDKNESASQGGAIFGKNCQISDNEGPVVFRDSTACLGGGAIAAQEIVSIQNNQAGISFEGGKASFGGGIACGSFSSAGGASVLGTIDISKNLGAISFSRTLCTTSDLGQMEYQGGGALFGENISLSENAGVLTFKDNIVKTFASNGKILGGGAILATGKVEITNNSEGISFTGNARAPQALPTQEEFPLFSKKEGRPLSSGYSGGGAILGREVAILHNAAVVFEQNRLQCSEEEATLLGCCGGGAVHGMDSTSIVGNSSVRFGNNYAMGQGVSGGALLSKTVQLAGNGSVDFSRNIASLGGGALQASEGNCELVDNGYVLFRDNRGRVYGGAISCLRGDVVISGNKGRVEFKDNIATRLYVEETVEKVEEVEPAPEQKDNNELSFLGRAEQSFITAANQALFASEDGDLSPESSISSEELAKRRECAGGAIFAKRVRIVDNQEAVVFSNNFSDIYGGAIFTGSLREEDKLDGQIPEVLISGNAGDVVFSGNSSKRDEHLPHTGGGAICTQNLTISQNTGNVLFYNNVACSGGAVRIEDHGNVLLEAFGGDIVFKGNSSFRAQGSDAIYFAGKESHITALNATEGHAIVFHDALVFENLEERKSAEVLLINSRENPGYTGSIRFLEAESKVPQCIHVQQGSLELLNGATLCSYGFKQDAGAKLVLAAGAKLKILDSGTPVQQGHAISKPEAEIESSSEPEGAHSLWIAKNAQTTVPMVDIHTISVDLASFSSSQQEGTVEAPQVIVPGGSYVRSGELNLELVNTTGTGYENHALLKNEAKVPLMSFVASGDEASAEISNLSVSDLQIHVVTPEIEEDTYGHMGDWSEAKIQDGTLVISWNPTGYRLDPQKAGALVFNALWEEGAVLSALKNARFAHNLTAQRMEFDYSTNVWGFAFGGFRTLSAENLVAIDGYKGAYGGASAGVDIQLMEDFVLGVSGAAFLGKMDSQKFDAEVSRKGVVGSVYTGFLAGSWFFKGQYSLGETQNDMKTRYGVLGESSASWTSRGVLADALVEYRSLVGPVRPTFYALHFNPYVEVSYASMKFPGFTEQGREARSFEDASLTNITIPLGMKFELAFIKGQFSEVNSLGISYAWEAYRKVEGGAVQLLEAGFDWEGAPMDLPRQELRVALENNTEWSSYFSTVLGLTAFCGGFTSTDSKLGYEANTGLRLIF

SEQ ID NO: 27 - protein, 2892 aa, Chlamydia trachomatis
ATGAAAAAAGCGTTTTTCTTTTTCCTTATCGGAAACTCCCTATCAGGACTAGCTAGAGAGGTTCCTTCTAGAATCTTTCTTATGCCCAACTCAGTTCCAGATCCTACGAAAGAGTCGCTATCAAATAAAATTAGTTTGACAGGAGACACTCACAATCTCACTAACTGCTATCTCGATAACCTACGCTACATACTGGCTATTCTACAAAAAACTCCCAATGAAGGAGCTGCTGTCACAATAACAGATTACCTAAGCTTTTTTGATACACAAAAAGAAGGTATTTATTTTGCAAAAAATCTCACCCCTGAAAGTGGTGGTGCGATTGGTTATGCGAGTCCCAATTCTCCTACCGTGGAGATTCGTGATACAATAGGTCCTGTAATCTTTGAAAATAATACTTGTTGCAGACTATTTACATGGAGAAATCCTTATGCTGCTGATAAAATAAGAGAAGGCGGAGCCATTCATGCTCAAAATCTTTACATAAATCATAATCATGATGTGGTCGGATTTATGAAGAACTTTTCTTATGTCCAAGGAGGAGCCATTAGTACCGCTAATACCTTTGTTGTGAGCGAGAATCAGTCTTGTTTTCTCTTTATGGACAACATCTGTATTCAAACTAATACAGCAGGAAAAGGTGGCGCTATCTATGCTGGAACGAGCAATTCTTTTGAGAGTAATAACTGCGATCTCTTCTTCATCAATAACGCCTGTTGTGCAGGAGGAGCGATCTTCTCCCCTATCTGTTCTCTAACAGGAAATCGTGGTAACATCGTTTTCTATAACAATCGCTGCTTTAAAAATGTAGAAACAGCTTCTTCAGAAGCTTCTGATGGAGGAGCAATTAAAGTAACTACTCGCCTAGATGTTACAGGCAATCGTGGTAGGATCTTTTTTAGTGACAATATCACAAAAAATTATGGCGGAGCTATTTACGCTCCTGTAGTTACCCTAGTGGATAATGGCCCTACCTACTTTATAAACAATATCGCCAATAATAAGGGGGGCGCTATCTATATAGACGGAACCAGTAACTCCAAAATTTCTGCCGACCGCCATGCTATTATTTTTAATGAAAATATTGTGACTAATGTAACTAATGCAAATGGTACCAGTACGTCAGCTAATCCTCCTAGAAGAAATGCAATAACAGTAGCAAGCTCCTCTGGTGAAATTCTATTAGGAGCAGGGAGTAGCCAAAATTTAATTTTTTATGATCCTATTGAAGTTAGCAATGCAGGGGTCTCTGTGTCCTTCAATAAGGAAGCTGATCAAACAGGCTCTGTAGTATTTTCAGGAGCTACTGTTAATTCTGCAGATTTTCATCAACGCAATTTACAAACAAAAACACCTGCACCCCTTACTCTCAGTAATGGTTTTCTATGTATCGAAGATCATGCTCAGCTTACAGTGAATCGATTCACACAAACTGGGGGTGTTGTTTCTCTTGGGAATGGAGCAGTTCTGAGTTGCTATAAAAATGGTACAGGAGATTCTGCTAGCAATGCCTCTATAACACTGAAGCATATTGGATTGAATCTTTCTTCCATTCTGAAAAGTGGTGCTGAGATTCCTTTATTGTGGGTAGAGCCTACAAATAACAGCAATAACTATACAGCAGATACTGCAGCTACCTTTTCATTAAGTGATGTAAAACTCTCACTCATTGATGACTACGGGAACTCTCCTTATGAATCCACAGATCTGACCCATGCTCTGTCATCACAGCCTATGCTATCTATTTCTGAAGCTAGCGATAACCAGCTACAATCAGAAAATATAGATTTTTCGGGACTAAATGTCCCTCATTATGGATGGCAAGGACTTTGGACTTGGGGCTGGGCAAAAACTCAAGATCCAGAACCAGCATCTTCAGCAACAATCACTGATCCACAAAAAGCCAATAGATTTCATAGAACCTTACTACTAACATGGCTTCCTGCCGGGTATGTTCCTAGCCCAAAACACAGAAGTCCCCTCATAGCTAACACCTTATGGGGGAATATGCTGCTTGCAACAGAAAGCTTAAAAAATAGTGCAGAGCTGACACCTAGTGGTCATCCTTTCTGGGGAATTACAGGAGGAGGACTAGGCATGATGGTTTACCAAGATCCTCGAGAAAATCATCCTGGATTCCATATGCGCTCTTCCGGATACTCTGCGGGGATGATAGCAGGGCAGACACACACCTTCTCATTGAAATTCAGTCAGACCTACACCAAACTCAATGAGCGTTACGCAAAAAACAACGTATCTTCTAAAAATTACTCATGCCAAGGAGAAATGCTCTTCTCATTGCAAGAAGGTTTCTTGCTGACTAAATTAGTTGGGCTTTACAGCTATGGAGACCATAACTGTCACCATTTCTATACTCAAGGAGAAAATCTAACATCTCAAGGGACGTTCCGCAGTCAAACGATGGGAGGTGCTGTCTTTTTTGATCTCCCTATGAAACCCTTTGGATCAACGCATATACTGACAGCTCCCTTTTTAGGTGCTCTTGGTATTTATTCTAGCCTGTCTCACTTTACTGAGGTGGGAGCCTATCCGCGAAGCTTTTCTACAAAGACTCCTTTGATCAATGTCCTAGTCCCTATTGGAGTTAAAGGTAGCTTTATGAATGCTACCCACAGACCTCAAGCCTGGACTGTAGAATTGGCATACCAACCCGTTCTGTATAGACAAGAACCAGGGATCGCAGCCCAGCTCCTAGCCAGTAAGGGTATTTGGTTCGGTAGTGGAAGCCCCTCATCGCGTCATGCCATGTCCTATAAAATCTCACAGCAAACACAACCTTTGAGTTGGTTAACTCTCCATTTCCAGTATCATGGATTCTACTCCTCTTCAACCTTCTGTAATTATCTCAATGGGGAAATTGCTCTGCGATTC

SEQ ID NO: 28 - protein, 1034 aa, Chlamydia trachomatis
MIKRTSLSFACLSFFYLSTISILQANETDTLQFRRFTFSDREIQFVLDPASLITAQNIVLSNLQSNGTGACTISGNTQTQIFSNSVNTTADSGGAFDMVTTSFTASDNANLLFCNNYCTHNKGGGAIRSGGPIRFLNNQDVLFYNNISAGAKYVGTGDHNEKNRGGALYATTITLTGNRTLAFINNMSGDCGGAISADTQISITDTVKGILFENNHTLNHIPYTQAENMARGGAICSRRDLCSISNNSGPIVFNYNQGGKGGAISATRCVIDNNKERIIFSNNSSLGWSQSSSASNGGAIQTTQGFTLRNNKGSIYFDSNTATHAGGAINCGYIDIRDNGPVYFLNNSAAWGAAFNLSKPRSATNYIHTGTGDIVFNNNVVFTLDGNLLGKRKLFHINNNEITPYTLSLGAKKDTRIYFYDLFQWERVKENTSNNPPSPTSRNTITVNPETEFSGAVVFSYNQMSSDIRTLMGKEHNYIKEAPTTLKFGTLAIEDDAELEIFNIPFTQNPTSLLALGSGATLTVGKHGKLNITNLGVILPIILKEGKSPPCIRVNPQDMTQNTGTGQTPSSTSSISTPMIIFNGRLSIVDENYESVYDSMDLSRGKAEQLILSIETTNDGQLDSNWQSSLNTSLLSPPHYGYQGLWTPNWITTTYTITLNNNSSAPTSATSIAEQKKTSETFTPSNTTTASIPNIKASAGSGSGSASNSGEVTITKHTLVVNWAPVGYIVDPIRRGDLIANSLVHSGRNMTMGLRSLLPDNSWFALQGAATTLFTKQQKRLSYHGYSSASKGYTVSSQASGAHGHKFLLSFSQSSDKMKEKETNNRLSSRYYLSALCFEHPMFDRIALIGAAACNYGTHNMRSFYGTKKSSKGKFHSTTLGASLRCELRDSMPLRSIMLTPFAQALFSRTEPASIRESGDLARLFTLEQAHTAVVSPIGIKGAYSSDTWPTLSWEMELAYQPTLYWKRPLLNTLLIQNNGSWVTTNTPLAKHSFYGRGSHSLKFSHLKLFANYQAEVATSTVSHYINAGGALVF

SEQ ID NO: 29 - protein, 1013 aa, Chlamydia trachomatis
MQTSFHKFFLSMILAYSCCSLSGGGYAAEIMIPQGIYDGETLTVSFPYTVIGDPSGTTVFSAGELTLKNLDNSIAALPLSCFGNLLGSFTVLGRGHSLTFENIRTSTNGAALSDSANSGLFTIEGFKELSFSNCNSLLAVLPAATTNNGSQTPTTTSTPSNGTIYSKTDLLLLNNEKFSFYSNLVSGDGGAIDAKSLTVQGISKLCVFQENTAQADGGACQVVTSFSAMANEAPIAFIANVAGVRGGGIAAVQDGQQGVSSSTSTEDPVVSFSRNTAVEFDGNVARVGGGIYSYGNVAFLNNGKTLFLNNVASPVYIAAEQPTNGQASNTSDNYGDGGAIFCKNGAQAAGSNNSGSVSFDGEGVVFFSSNVAAGKGGAIYAKKLSVANCGPVQFLGNIANDGGAIYLGESGELSLSADYGDIIFDGNLKRTAKENAADVNGVTVSSQAISMGSGGKITTLRAKAGHQILFNDPIEMANGNNQPAQSSEPLKINDGEGYTGDIVFANGNSTLYQNVTIEQGRIVLREKAKLSVNSLSQTGGSLYMEAGSTLDFVTPQPPQQPPAANQLITLSNLHLSLSSLLANNAVTNPPTNPPAQDSHPAIIGSTTAGSVTISGPIFFEDLDDTAYDRYDWLGSNQKIDVLKLQLGTQPSANAPSDLTLGNEMPKYGYQGSWKLAWDPNTANNGPYTLKATWTKTGYNPGPERVASLVPNSLWGSILDIRSAHSAIQASVDGRSYCRGLWVSGVSNFFYHDRDALGQGYRYISGGYSLGANSYFGSSMFGLAFTEVFGRSKDYVVCRSNHHACIGSVYLSTKQALCGSYLFGDAFIRASYGFGNQHMKTSYTFAEESDVRWDNNCLVGEIGVGLPIVITPSKLYLNELRPFVQAEFSYADHESFTEEGDQARAFRSGHLMNLSVPVGVKFDRCSSTHPNKYSFMGAYICDAYRTISGTQTTLLSHQETWTTDAFHLARHGVIVRGSMYASLTSNIEVYGHGRYEYRDTSRGYGLSAGSKVRF

SEQ ID NO: 30 - protein, 1016 aa, Chlamydia trachomatis
MPFSLRSTSFCFLACLCSYSYGFASSPQVLTPNVTTPFKGDDVYLNGDCAFVNVYAGAENGSIISANGDNLTITGQNHTLSFTDSQGPVLQNYAFISAGETLTLKDFSSLMFSKNVSCGEKGMISGKTVSISGAGEVIFWDNSVGYSPLSIVPASTPTPPAPAPAPAASSSLSPTVSDARKGSIFSVETSLEISGVKKGVMFDNNAGNFGTVFRGNSNNNAGSGGSGSATTPSFTVKNCKGKVSFTDNVASCGGGVVYKGTVLFKDNEGGIFFRGNTAYDDLGILAATSRDQNTETGGGGGVICSPDDSVKFEGNKGSIVFDYNFAKGRGGSILTKEFSLVADDSVVFSNNTAEKGGGAIYAPTIDISTNGGSILFERNRAAEGGAICVSEASSGSTGNLTLSASDGDIVFSGNMTSDRPGERSAARILSDGTTVSLNASGLSKLIFYDPVVQNNSAAGASTPSPSSSSMPGAVTINQSGNGSVIFTAESLTPSEKLQVLNSTSNFPGALTVSGGELVVTEGATLTTGTITATSGRVTLGSGASLSAVAGAANNNYTCTVSKLGIDLESFLTPNYKTAILGADGTVTVNSGSTLDLVMESEAEVYDNPLFVGSLTIPFVTLSSSSASNGVTKNSVTINDADAAHYGYQGSWSADWTKPPLAPDAKGMVPPNTNNTLYLTWRPASNYGEYRLDPQRKGELVPNSLWVAGSALRTFTNGLKEHYVSRDVGFVASLHALGDYILNYTQDDRDGFLARYGGFQATAASHYENGSIFGVAFGQLYGQTKSRMYYSKDAGNMTMLSCFGRSYVDIKGTETVMYWETAYGYSVHRMHTQYFNDKTQKFDHSKCHWHNNNYYAFVGAEHNFLEYCIPTRQFARDYELTGFMRFEMAGGWSSSTRETGSLTRYFARGSGHNMSLPIGIVAHAVSHVRRSPPSKLTLNMGYRPDIWRVTPHCNMEIIANGVKTPIQGSPLARHAFFLEVHDTLYIHHFGRAYMNYSLDARRRQTAHFVSMGLNRIF

SEQ ID NO: 31 - protein, 878 aa, Chlamydia trachomatis
MRPDHMNFCCLCAAILSSTAVLFGQDPLGETALLTKNPNHVVCTFFEDCTMESLFPALCAHASQDDPLYVLGNSYCWFVSKLHITDPKEALFKEKGDLSIQNFRFLSFTDCSSKESSPSIIHQKNGQLSLRNNGSMSFCRNHAEGSGGAISADAFSLQHNYLFTAFEENSSKGNGGAIQAQTFSLSRNVSPISFARNRADLNGGAICCSNLICSGNVNPLFFTGNSATNGGAICCISDLNTSEKGSLSLACNQETLFASNSAKEKGGAIYAKHMVLRYNGPVSFINNSAKIGGAIAIQSGGSLSILAGEGSVLFQNNSQRTSDQGLVRNAIYLEKDAILSSLEARNGDILFFDPIVQESSSKESPLPSSLQASVTSPTPATASPLVIQTSANRSVIFSSERLSEEEKTPDNLTSQLQQPIELKSGRLVLKDRAVLSAPSLSQDPQALLIMEAGTSLKTSSDLKLATLSIPLHSLDTEKSVTIHAPNLSIQKIFLSNSGDENFYENVELLSKEQNNIPLLTLSKEQSHLHLPDGNLSSHFGYQGDWTFSWKDSDEGHSLIANWTPKNYVPHPERQSTLVANTLWNTYSDMQAVQSMINTIAHGGAYLFGTWGSAVSNLFYAHDSSGKPIDNWHHRSLGYLFGISTHSLDDHSFCLAAGQLLGKSSDSFITSTETTSYIATVQAQLATPLMKISAQACYNESIHELKTKYRSFSKEGFGSWHSVAVSGEVCASIPIVSNGSGLFSSFSIFSKLQGFSGTQDGFEESSGEIRSFSASSFRNISLPMGITFEKKSQKTRNYYYFLGAYIQDLKRDVESGPVVLLKNAVSWDAPMANLDSRAYMFRLTNQRALHRLQTLLNVSYVLRGQSHSYSLDLGTTYRF

SEQ ID NO: 32 - protein, 412 aa, Chlamydia trachomatis
MQQLIDNLKKRGILDNSSAGLESLTVPVSAYLGFDPTAPSLHIGHWIGICFLRRLAAYGITPVALVGGATGMIGDPSGKSVERSLLDQAQVLDNSKKIAAALASYLPGIRIVNNADWLGSLSMVDFLRDVGKHFRLGSMLAKDVVKQRVYSEEGISYTEFSYLLLQSYDFAHLFKEHNVVLQCGGSDQWGNITSGIDYIRRRGLGQAYGLTYPLLTDSKGKKIGKTESGTIWLDPALTPPYELFQYFLRLPDQEISKVMRTLTLLDNEEIFALDERLTSDPQAVKKYIAEVIVKDVHGSEGLAQAQAATESFFASKGKSITEAELVALVESGVGVKVARADLIGKRWLDIVVELGFCSSRGQARRLIQQRGLYINQEPLADEQSILDGTQLCFDRYVLLSQGKRKKQVIDLN

SEQ ID NO: 33 - protein, 188 aa, Chlamydia trachomatis
MRKTIFKAFNLLFSLLFLSSCSYPCRDWECHGCDSARPRKSSFGFVPFYSDEEIQQAFVEDFDSKEEQLYKTSAQSTSFRNITFATDSYSIKGEDNLTILASLVRHLHKSPKATLYIEGHTDERGAAAYNLALGARRANAVKQYLIKQGIAADRLFTISYGKEHPVHPGHNELAWQQNRRTEFKIHAR

SEQ ID NO: 34 - protein, 20 aa, Chlamydia trachomatis
PKATLYIEGHTDERGAAAYN

SEQ ID NO: 35 - protein, 18 aa, Chlamydia trachomatis
EYIVSGNASFTKFTNIPT

SEQ ID NO: 36 - protein, 19 aa, Chlamydia trachomatis
QLYLGPFWTLYGNYTIDVG

SEQ ID NO: 37 - protein, 21 aa, Chlamydia trachomatis
SFIKTLNSVGSTVNQLNKPLS

(19) United States
(12) Patent Application Publication
(10) Pub. No.: US 2013/0095487 A1
(43) Pub. Date: Apr. 18, 2013

(54) Interferon-Gamma Response as a Diagnostic Test for Persistent Chlamydial Infections

(71) Applicants: University of South Florida, Tampa, FL (US); University Health Network, Toronto (CA); Wayne State University, Detroit, MI (US)

(72) Inventors: John D. Carter, Tampa, FL (US); Alan P. Hudson, Detroit, MI (US); Robert D. Inman, Toronto (CA)

(73) Assignees: UNIVERSITY OF SOUTH FLORIDA, Tampa, FL (US); WAYNE STATE UNIVERSITY, Detroit, MI (US); UNIVERSITY HEALTH NETWORK, Toronto (CA)

(21) Appl. No.: 13/655,038

(22) Filed: Oct. 18, 2012

Related U.S. Application Data
(60) Provisional application No. 61/548,418, filed on Oct. 18, 2011.

(51) Int. Cl. G01N 33/571; G01N 21/64; C12M 1/34; C12Q 1/68

(52) U.S. Cl. 435/6.11; 435/7.1; 435/7.92; 435/6.12; 435/287.2

(57) ABSTRACT

The present invention provides a non-invasive, sensitive, and convenient diagnostic test for persistent Chlamydial infection and diseases arising from persistent Chlamydial infection. The present invention also provides kits for diagnosis of persistent Chlamydial infection.

### CROSS-REFERENCE TO RELATED APPLICATION

This application claims the benefit of U.S. provisional application Ser. No. 61/548,418, filed Oct. 18, 2011, which is herein incorporated by reference in its entirety.

### BACKGROUND OF THE INVENTION

Chlamydia trachomatis is a prevalent sexually transmitted gram-positive bacteria species that causes an estimated 3 million new cases of genital infections annually in the United States. Prospective studies indicate that patients with acute, active Chlamydia trachomatis infection in the genital areas may develop chronic, persistent infection, during which Chlamydia trachomatis migrates into distant areas such as synovial tissues, ocular tissues, and blood. Specifically, about 5% of patients with acute Chlamydia trachomatis infection (or as many as 150,000 cases) develop Chlamydia-induced reactive arthritis (ReA). This number likely represents a low estimate of about half or even fewer of the total ReA cases, as cases of ReA induced by persistent Chlamydia pneumoniae (Cpn) infection have not been included. Diseases that can arise from persistent Chlamydial infection also include, for example, undifferentiated Spondyloarthritis (uSpA), trachoma, female infertility, coronary artery disease, asthma, and certain types of cervical cancer.

Currently, there lacks a validated, commercially available diagnostic test for persistent Chlamydial infections, in general, or Chlamydia-induced ReA, in particular. The most accurate diagnostic test for ReA is based on PCR analyses of biopsy samples containing infected synovial tissue. However, this PCR-based diagnostic test suffers several limitations. First, one must perform a synovial biopsy, which is an invasive procedure very difficult to apply in everyday practice. Further, PCR interpretation is a learned science and few laboratories are equipped to accurately analyze synovial tissue in such a manner. As a result, a myriad of diseases associated with persistent Chlamydial infection, including Chlamydia-induced ReA, remain vastly under-diagnosed. This, in turn, leads to less efficacious treatment, particularly if an adequate therapy exists but the condition goes undiagnosed. Therefore, there is a substantial need for an improved diagnostic test for persistent Chlamydial infections.

### BRIEF SUMMARY OF THE INVENTION

The aforementioned need is satisfied by the present invention, providing non-invasive, sensitive, and convenient diagnostic methods for persistent Chlamydial infection and diseases arising from persistent Chlamydial infection.

In preferred embodiments, persistent Chlamydial infection can be detected by a single whole blood assay. Specifically, the level of INF-γ in blood samples in response to antigenic stimulation by peptide antigens that are specific for persistent Chlamydial infection is determined. The elevation of the INF-γ level, as compared to the predetermined reference value, is diagnostic of persistent Chlamydial infection.

In an embodiment, the method for diagnosing persistent Chlamydial infection comprises:

a) obtaining a biological sample from a subject suspected of having persistent Chlamydial infection, wherein the biological sample contains immune cells capable of producing IFN-γ;

b) contacting the biological sample with a peptide antigen;

c) determining a level of IFN-γ in the biological sample; and

d) comparing the level of IFN-γ in the sample to a predetermined reference value;

wherein the peptide antigen is derived from Chlamydial HSP-60 protein or other Chlamydial proteins expressed during persistent infection; and

wherein an elevated level of IFN-γ in the biological sample compared to the predetermined reference value indicates that the subject has persistent Chlamydial infection.

Advantageously, the present diagnostic method can be used to detect diseases arising from, or associated with, persistent Chlamydial infection, such as for example, Chlamydia-induced reactive arthritis, undifferentiated Spondyloarthritis (uSpA), trachoma, female infertility, coronary artery disease, asthma, and certain types of cervical cancer.

The present invention also provides kits for diagnosis of persistent Chlamydial infection. The kit comprises peptide antigen molecules derived from Chlamydial HSP-60 protein or other proteins expressed during persistent infection. Also provided are methods for generation of the peptide antigens of the present invention and methods for treating persistent Chlamydial infection.

### BRIEF DESCRIPTION OF THE DRAWINGS

FIG. 1 shows relative interferon-gamma levels of monocytes incubated with peptide antigens (CT600, CT413-2, CT413-3, CT849) that are unique and specific to persistent Chlamydial infections.

FIG. 2 illustrates a study design of combination antibiotic therapy for persistent Chlamydial infection.

FIG. 3 illustrates a study design of combination antibiotic therapy for persistent Chlamydial infection.

### BRIEF DESCRIPTION OF THE SEQUENCES

SEQ ID NO: 1 is an amino acid sequence of Chlamydia trachomatis (Ct) protein encoded by Ct604.

SEQ ID NO: 2 is an amino acid sequence of Chlamydia trachomatis (Ct) protein encoded by Ct110.

SEQ ID NO: 3 is an amino acid sequence of Chlamydia trachomatis (Ct) protein encoded by Ct755.

SEQ ID NO: 4 is an amino acid sequence of Chlamydia trachomatis (Ct) protein encoded by Ct135.

SEQ ID NO: 5 is an amino acid sequence of Chlamydia trachomatis (Ct) protein encoded by Ct166 (toxB-related protein).

SEQ ID NO: 6 is an amino acid sequence of Chlamydia trachomatis (Ct) protein encoded by Ct849.

SEQ ID NO: 7 is an amino acid sequence of Chlamydia trachomatis (Ct) protein encoded by Ct552.

SEQ ID NO: 8 is an amino acid sequence of Chlamydia trachomatis (Ct) protein encoded by Ct666.

SEQ ID NO: 9 is an amino acid sequence of Chlamydia trachomatis (Ct) protein encoded by Ct173.

SEQ ID NO: 10 is an amino acid sequence of Chlamydia trachomatis (Ct) protein encoded by Ct769.

SEQ ID NO: 11 is an amino acid sequence of Chlamydia trachomatis (Ct) protein encoded by Ct172.1.

SEQ ID NO: 12 is an amino acid sequence of Chlamydia trachomatis (Ct) protein encoded by Ct321.

SEQ ID NO: 13 is an amino acid sequence of Chlamydia trachomatis (Ct) protein encoded by Ct456.

SEQ ID NO: 14 is an amino acid sequence of Chlamydia trachomatis (Ct) protein encoded by Ct875.

SEQ ID NO: 15 is an amino acid sequence of Chlamydia trachomatis (Ct) protein encoded by Ct659.

SEQ ID NO: 16 is an amino acid sequence of Chlamydia trachomatis (Ct) protein encoded by Ct326.1.

SEQ ID NO: 17 is an amino acid sequence of Chlamydia trachomatis (Ct) protein encoded by Ct229.

SEQ ID NO: 18 is an amino acid sequence of Chlamydia trachomatis (Ct) protein encoded by Ct657.

SEQ ID NO: 19 is an amino acid sequence of Chlamydia trachomatis (Ct) protein encoded by Ct143.

SEQ ID NO: 20 is an amino acid sequence of Chlamydia trachomatis (Ct) protein encoded by Ct728.

SEQ ID NO: 21 is an amino acid sequence of Chlamydia trachomatis (Ct) protein encoded by Ct051.

SEQ ID NO: 22 is an amino acid sequence of Chlamydia trachomatis (Ct) protein encoded by Ct136.

SEQ ID NO: 23 is an amino acid sequence of Chlamydia trachomatis (Ct) protein encoded by Ct412.

SEQ ID NO: 24 is an amino acid sequence of Chlamydia trachomatis (Ct) protein encoded by Ct413.

SEQ ID NO: 25 is an amino acid sequence of Chlamydia trachomatis (Ct) protein encoded by Ct414.

SEQ ID NO: 26 is an amino acid sequence of Chlamydia trachomatis (Ct) protein encoded by Ct812.

SEQ ID NO: 27 is an amino acid sequence of Chlamydia trachomatis (Ct) protein encoded by Ct869.

SEQ ID NO: 28 is an amino acid sequence of Chlamydia trachomatis (Ct) protein encoded by Ct870.

SEQ ID NO: 29 is an amino acid sequence of Chlamydia trachomatis (Ct) protein encoded by Ct871.

SEQ ID NO: 30 is an amino acid sequence of Chlamydia trachomatis (Ct) protein encoded by Ct872.

SEQ ID NO: 31 is an amino acid sequence of Chlamydia trachomatis (Ct) protein encoded by Ct874.

SEQ ID NO: 32 is an amino acid sequence of Chlamydia trachomatis (Ct) protein encoded by Ct062.

SEQ ID NO: 33 is an amino acid sequence of Chlamydia trachomatis (Ct) protein encoded by Ct600.

SEQ ID NO: 34 is the amino acid sequence of an antigen peptide derived from Chlamydia trachomatis (Ct) protein encoded by Ct600. The antigen peptide (CT600) of SEQ ID NO: 34 can be used in the diagnostic assay for persistent Chlamydial infection.

SEQ ID NO: 35 is the amino acid sequence of an antigen peptide derived from Chlamydia trachomatis (Ct) protein encoded by Ct413. The antigen peptide (CT413-2) of SEQ ID NO: 35 can be used in the diagnostic assay for persistent Chlamydial infection.

SEQ ID NO: 36 is the amino acid sequence of an antigen peptide derived from Chlamydia trachomatis (Ct) protein encoded by Ct413. The antigen peptide (CT413-3) of SEQ ID NO: 36 can be used in the diagnostic assay for persistent Chlamydial infection.

SEQ ID NO: 37 is the amino acid sequence of an antigen peptide derived from Chlamydia trachomatis (Ct) protein encoded by Ct849. The antigen peptide (CT849) of SEQ ID NO: 37 can be used in the diagnostic assay for persistent Chlamydial infection.

### DETAILED DISCLOSURE OF THE INVENTION

The present invention provides simple, non-invasive, and sensitive diagnostic methods for persistent Chlamydial infection and diseases arising from persistent Chlamydial infection. In preferred embodiments, persistent Chlamydial infection can be detected by a single whole blood assay. Specifically, the level of INF-γ in blood samples in response to antigenic stimulation by peptide antigens of the present invention is determined. The presence or elevation of the INF-γ level, as compared to the predetermined reference value, is diagnostic of persistent Chlamydial infection.

The present invention also provides kits for diagnosis of persistent Chlamydial infection. Also provided are methods for generation of the peptide antigens of the present invention and methods for treating persistent Chlamydial infection.

The present invention is based, at least in part, on the discovery that during persistent infection Chlamydiae exists in a morphologically aberrant but metabolically active state, and displays a gene expression profile significantly different from that observed during normal active infection. During persistence, the expression of the major outer membrane protein (omp1) gene and genes required for cell division are substantially down-regulated. In addition, Chlamydial heat shock protein-60 (HSP-60) homologs are differentially expressed. For instance, C. trachomatis (Ct) HSP-60 proteins can be encoded by three paralog genes Ct110, Ct604, and Ct755. During in vivo persistent infection of synovial tissue, Ct displays moderate up-regulation of Ct110, significant up-regulation of Ct604, and down-regulation of Ct755. Macrophages are primary host cells during in vivo persistent synovial Ct infection. Further, HSP-60 paralogs display different expression profiles when comparing in vitro with in vivo persistent infections. Ct110 expression is down-regulated in vitro in infected monocytes, but remains elevated in vivo in synovial cells. Ct604 expression is significantly up-regulated both in vitro and in vivo. Ct755 expression is down-regulated or even terminated both in vitro and in vivo during persistent infection.

Persistent Chlamydial (such as C. trachomatis and C. pneumoniae) infection induces host immune responses. Specifically, the T-helper cell 1 (Tₕ1)-mediated interferon-gamma (IFN-γ) response is heightened in patients with persistent Chlamydial infections. The lack of IFN-γ expression in Tₕ1 and Tₕ2 cells is associated with increased Chlamydial burden. IFN-γ is also involved in host responses against aberrant developmental life cycle of persistent forms of Chlamydiae. As optimal host IFN-γ-mediated T-cell response also requires the participation of other immune effector molecules such as IL-12p70, host IFN-γ responses can also be evidenced by changes in IL-12p70 levels. The present inventors have recently discovered that after antibiotic therapy, serum levels of IL-12p70 are significantly reduced in patients with persistent Chlamydial infection, evidencing IFN-γ's role in host immune responses during persistent infection.

The term “persistent bacterial infection,” as used herein, refers to its ordinary meaning that is an infection that is not completely eradicated through standard treatment regimens using anti-bacterial agents. Persistent bacterial infections are caused by bacteria capable of establishing a cryptic or latent phase of infection and may be classified as such by culturing the bacteria from a patient and demonstrating bacterial survival in vitro in the presence of anti-bacterial agents or by determination of anti-bacterial treatment failure in a patient.

It is contemplated by the present inventors that the unique transcription profile of Chlamydiae during persistence, which can be detected by measuring in vitro IFN-γ responses, is diagnostic of persistent Chlamydial infection. Specifically, Chlamydial HSP-60 proteins, which are differentially expressed during persistence, are highly immunogenic. As a result, patients with persistent Chlamydial infection have lymphocytes that recognize Chlamydial HSP-60 expressed during persistence in their blood. The lymphocytic recognition process involves the release of cytokines such as IFN-γ. Thus, the presence or elevation of IFN-γ level in the blood sample, in response to in vitro stimulation by Chlamydial HSP-60 peptide antigen, is diagnostic of persistent Chlamydial infection.

It is also contemplated that the present diagnostic test can be used to detect diseases associated with persistent Chlamydial infection, such as ReA. Chlamydiae have been shown to exist in the joint in a metabolically persistent state in patients with Chlamydia-induced arthritis. Using PCR analysis, Chlamydia DNA has been detected in synovial biopsies of ReA patients years after initial exposure to the bacteria. Clinical trials recently conducted by the inventors also revealed similar Chlamydial viability in synovial tissue as well as peripheral blood mononucleated cells (PBMCs) of patients with Chlamydia-induced ReA.

### Diagnosis of Persistent Chlamydial Infection

In a first aspect, the present invention provides methods for diagnosing persistent Chlamydial infection and diseases arising from persistent Chlamydial infection. Advantageously, the present diagnostic method is non-invasive, convenient, sensitive, specific, and reliable.

In an embodiment, the method for diagnosing persistent Chlamydial infection comprises:

a) obtaining a biological sample from a subject, wherein the biological sample contains immune cells capable of producing IFN-γ;

b) contacting the biological sample with a peptide antigen;

c) determining a level of IFN-γ in the biological sample; and

d) comparing the level of IFN-γ in the sample to a predetermined reference value;

wherein the peptide antigen is derived from Chlamydial HSP-60 protein or other Chlamydial proteins expressed during persistent infection; and

wherein an elevated level of IFN-γ in the biological sample compared to the predetermined reference value indicates that the subject has persistent Chlamydial infection.

In one embodiment, the subject is suspected of having persistent Chlamydial infection.

In one embodiment, the peptide antigen is derived from a Chlamydial protein (such as Chlamydial HSP-60 protein) expressed during persistent infection, but is not expressed during acute infection. In another embodiment, the peptide antigen is derived from a Chlamydial protein (such as Chlamydial HSP-60 protein) whose expression level is upregulated during persistent infection, when compared to the expression level during acute infection.

In an embodiment, whole blood obtained from a subject is divided into two samples. The blood samples are incubated with peptide antigen molecules derived from Chlamydial HSP-60 and nothing (control), respectively. After incubation for 16 to 24 hours, the level of IFN-γ is measured. The control IFN-γ level can be used to adjust background IFN-γ responses. An elevated level of IFN-γ in response to stimulation by Chlamydial HSP-60 peptide antigen, as compared to the predetermined reference value, indicates that the subject has persistent Chlamydial (e.g., C. trachomatis or C. pneumoniae) infection.

In a specific embodiment, whole blood obtained from a subject is divided into two samples. The blood samples are incubated with peptide antigen molecules derived from Ct604-encoded HSP-60 protein and nothing (control), respectively. After incubation for 16 to 24 hours, the level of IFN-γ is measured. The control IFN-γ level can be used to adjust background IFN-γ responses. An elevated level of IFN-γ in response to stimulation by peptide antigen derived from Ct604-encoded HSP-60 protein, as compared to the predetermined reference value, indicates that the subject has persistent C. trachomatis infection.

Additionally or alternatively, following stimulation by an antigen derived from a Chlamydial protein that is expressed, or whose expression is upregulated during persistent infection, in vitro responses of immune effector molecules other than IFN-γ may be used to diagnose persistent Chlamydial infection. In an embodiment, the method for diagnosing persistent Chlamydial infection comprises:

a) obtaining a biological sample from a subject, wherein the biological sample contains immune cells capable of producing an immune effector molecule;

b) contacting the biological sample with a peptide antigen;

c) determining a level of an immune effector molecule in the biological sample; and

d) comparing the level of the immune effector molecule in the biological sample to a predetermined reference value;

wherein the peptide antigen is derived from Chlamydial HSP-60 protein or other proteins expressed during persistent infection; and

wherein an elevated level of the immune effector molecule in the biological sample as compared to the predetermined reference value indicates that the subject has persistent Chlamydial infection.

Immune effector molecules useful for diagnosis of persistent Chlamydial infection and/or diseases arising from persistent Chlamydial infection include, for example, IFN-γ, EGF, GM-CSF, IL-1γ, TGF-α, VEGF, and IL-12p70. Suitable immune effector molecules also include IL-1, IL-2, IL-3, IL-5, IL-4, IL-6, IL-8, IL-10, IL-12, IL-13, IL-14, IL-17, IL-18, IL-23, IL-24, IL-25, IL27, IL-32, TNF-α, IFN-β, and IFN-α.

The term “subject,” as used herein, describes an organism, including mammals such as primates. Mammalian species that can benefit from the subject methods include, but are not limited to, apes, chimpanzees, orangutans, humans, monkeys; and domesticated and/or laboratory animals such as dogs, cats, horses, cattle, pigs, sheep, goats, chickens, mice, rats, guinea pigs, and hamsters. Typically, the subject is a human.

The biological sample includes, but is not limited to, a sample containing blood, tissues, cells, and/or biological fluids (e.g., saliva, tears) isolated from a subject. In preferred embodiments, biological samples are obtained from, or derived from, blood, including whole blood, plasma, serum, and blood cells such as peripheral blood mononucleated cells (PBMCs). One skilled in the art would realize that some samples would be more readily analyzed following a fractionation or purification procedure, for example, separation of whole blood into serum or plasma components.

Preferably, biological samples contain immune cells, such as T cells, T helper cells, B cells, macrophages, monocytes, natural killer (NK) cells, dendritic cells, and other cells that are capable of releasing immune effector molecules (e.g., IFN-γ) in response to stimulation by Chlamydia-derived peptide antigens.

In an embodiment, the biological sample is not a urine sample. In another embodiment, the biological sample is not a synovial tissue sample. In an embodiment, the biological sample is not a biopsy sample.

In certain embodiments, peptide antigens of the present invention are derived from Chlamydial HSP-60 proteins expressed during persistent infection. In certain specific embodiments, peptide antigens of the present invention are derived from Chlamydial proteins whose expression is upregulated during persistent infection. In certain embodiments, peptide antigens can comprise the full length, or an immunogenic fragment of, Chlamydial HSP-60 protein. Preferably, peptide antigens do not share sequence identity or similarity to the full length or fragments of HSP-60 proteins expressed during acute Chlamydial infection.

In certain embodiments, the peptide antigen is an immunogenic fragment of, or otherwise derived from, the Chlamydia trachomatis (Ct) 604-encoded-HSP-60 protein homolog (SEQ ID NO:1). Preferably, the peptide antigen is not a fragment of, or otherwise derived from, amino acid residues 112-170 and 474-497 of the Ct604-encoded HSP-60 homolog (SEQ ID NO:1). In another embodiment, the peptide antigen is derived from Chlamydia pneumoniae (Cpn) HSP-60 expressed during persistent infection.

In certain embodiments, the peptide antigen is an immunogenic fragment of, or otherwise derived from, Chlamydia trachomatis (Ct) protein encoded by Ct604, Chlamydia trachomatis (Ct) protein encoded by Ct110, Chlamydia trachomatis (Ct) protein encoded by Ct755, Chlamydia trachomatis (Ct) protein encoded by Ct135, Chlamydia trachomatis (Ct) protein encoded by Ct166 (toxB-related protein), Chlamydia trachomatis (Ct) protein encoded by Ct849, Chlamydia trachomatis (Ct) protein encoded by Ct552, Chlamydia trachomatis (Ct) protein encoded by Ct666, Chlamydia trachomatis (Ct) protein encoded by Ct173, Chlamydia trachomatis (Ct) protein encoded by Ct769, Chlamydia trachomatis (Ct) protein encoded by Ct172.1, Chlamydia trachomatis (Ct) protein encoded by Ct321, Chlamydia trachomatis (Ct) protein encoded by Ct456, Chlamydia trachomatis (Ct) protein encoded by Ct875, Chlamydia trachomatis (Ct) protein encoded by Ct659, Chlamydia trachomatis (Ct) protein encoded by Ct326.1, Chlamydia trachomatis (Ct) protein encoded by Ct229, Chlamydia trachomatis (Ct) protein encoded by Ct657, Chlamydia trachomatis (Ct) protein encoded by Ct143, Chlamydia trachomatis (Ct) protein encoded by Ct728, Chlamydia trachomatis (Ct) protein encoded by Ct051, Chlamydia trachomatis (Ct) protein encoded by Ct136, Chlamydia trachomatis (Ct) protein encoded by Ct412, Chlamydia trachomatis (Ct) protein encoded by Ct413, Chlamydia trachomatis (Ct) protein encoded by Ct414, Chlamydia trachomatis (Ct) protein encoded by Ct812, Chlamydia trachomatis (Ct) protein encoded by Ct869, Chlamydia trachomatis (Ct) protein encoded by Ct870, Chlamydia trachomatis (Ct) protein encoded by Ct871, Chlamydia trachomatis (Ct) protein encoded by Ct872, Chlamydia trachomatis (Ct) protein encoded by Ct874, Chlamydia trachomatis (Ct) protein encoded by Ct062, and Chlamydia trachomatis (Ct) protein encoded by Ct600.

In certain embodiments, the peptide antigen is an immunogenic fragment of, or otherwise derived from, a Chlamydia trachomatis (Ct) protein having the amino acid sequence selected from SEQ ID NOs: 1-33.

In certain embodiments, the peptide antigen is an immunogenic fragment of, or otherwise derived from, Chlamydia trachomatis (Ct) 604-encoded protein (SEQ ID NO:1), Chlamydia trachomatis (Ct) 110-encoded protein (SEQ ID NO:2), Chlamydia trachomatis (Ct) 413-encoded protein (SEQ ID NO:24), Chlamydia trachomatis (Ct) 600-encoded protein (SEQ ID NO:33), or Chlamydia trachomatis (Ct) 849-encoded protein (SEQ ID NO:6).

In certain embodiments, peptide antigens of the present invention include the full length, or a fragment of, Chlamydial proteins selected from the group consisting of SEQ ID NOs:1-33.

In certain embodiments, the peptide antigen comprises any of SEQ ID NOs: 34-37.

Unless otherwise specified, as used herein, percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

While peptide antigens of the present invention can be of any length, they are preferably 6 to 50 amino acids in length or in any length between 10 to 50 amino acids, including for example, 8 to 40 amino acids in length, 8 to 30 amino acids in length, 8 to 20 amino acids in length, and 8 to 25 amino acids in length.

To avoid false positive diagnostic results, in an embodiment, peptide antigens of the present invention do not elicit immune responses (e.g., IFN-γ responses) in biological samples obtained from subjects with acute Chlamydial infection. Alternatively, peptide antigens of the present invention elicit different levels of immune responses in samples with persistent Chlamydial infection, as compared to samples with acute Chlamydial infection. In another embodiment, peptide antigens of the present invention do not elicit immune responses (e.g., IFN-γ responses) in biological samples obtained from subjects who have non-Chlamydial infection (e.g., bacterial, viral, fungal and other microbial infection), inflammation, or auto-immune diseases. Alternatively, peptide antigens of the present invention elicit different levels of immune responses in samples with persistent Chlamydial infection, as compared to samples obtained from subjects who have non-Chlamydial infection (e.g., bacterial, viral, fungal and other microbial infection), inflammation, or auto-immune diseases. Preferably, the biological sample is contacted or incubated with peptide antigen molecules for about 1-48 hours or any time periods in between, such as about 5 to 36 hours, about 8 to 30 hours, about 8 to 24 hours, or about 16 to 24 hours. In addition, the biological sample is preferably contacted or incubated with peptide antigen molecules at about 15° C. to 40° C. or any temperatures in between, such as about 25° C. to 37° C., about 28° C. to 37° C. or about 30° C. to 37° C.

Determination of levels or concentrations of immune effector molecules (such as IFN-γ) can be made at protein or nucleic acid (e.g., mRNA) levels. Such determination can be made using conventional methods, including but not limited to, enzyme-linked immunosorbant assay (ELISA), Western blot, immunoprecipitation, immunofluorescence, radioimmunoassay, immunocytochemistry, polymerase chain reaction (PCR) methods including reverse transcription polymerase chain reaction (RT-PCR), enzyme-linked immunosorbent spot (ELISpot) assay, Northern blot, nucleic acid hybridization techniques, fluorescent polarization (FO) technology, nucleic acid amplification techniques, transcription mediated amplification (TMA), DNA strand displacement amplification (SDA), or a combination thereof.

A contacting step in the assay (method) of the invention can involve contacting, combining, or mixing the biological sample and a solid support, such as a reaction vessel, microbeads, microvessel, tube, microtube, well, multi-well plate, or other solid support.

The level of an immune effector molecule (such as IFN-γ) can be determined based on protein level. In one embodiment, the level of an immune effector molecule (such as IFN-γ) can be determined by contacting an antibody, aptamer, or binding partner that specifically binds to the immune effector molecule (such as IFN-γ). An antibody that specifically recognizes, or specifically binds to, immune effector molecules (such as IFN-γ) can be in any of a variety of forms, including intact immunoglobulin molecules, fragments of immunoglobulin molecules such as Fv, Fab and similar fragments; multimers of immunoglobulin molecules (e.g., diabodies, triabodies, and bi-specific and tri-specific antibodies, as are known in the art; see, e.g., Hudson and Kortt, J. Immunol. Methods 231:177 189, 1999); fusion constructs containing an antibody or antibody fragment; and human or humanized immunoglobulin molecules or fragments thereof.

“Specific binding” or “specificity” refers to the ability of a protein to detectably bind an epitope presented on a protein or polypeptide molecule of interest, while having relatively little detectable reactivity with other proteins or structures. Specificity can be relatively determined by binding or competitive binding assays, using, e.g., Biacore instruments. Specificity can be exhibited by, e.g., an about 10:1, about 20:1, about 50:1, about 100:1, 10.000:1 or greater ratio of affinity/avidity in binding to the specific target molecule versus nonspecific binding to other irrelevant molecules.

Antibodies within the scope of the invention can be of any isotype, including IgG, IgA, IgE, IgD, and IgM. IgG isotype antibodies can be further subdivided into IgG1, IgG2, IgG3, and IgG4 subtypes. IgA antibodies can be further subdivided into IgA1 and IgA2 subtypes.

Antibodies useful according to the present invention include polyclonal and monoclonal antibodies. The term “monoclonal antibody,” as used herein, refers to an antibody or antibody fragment obtained from a substantially homogeneous population of antibodies or antibody fragments (i.e. the individual antibodies within the population are identical except for possible naturally occurring mutations that may be present in a small subset of the antibody molecules).

In one embodiment, the level of an immune effector molecule (such as IFN-γ) is determined by contacting the biological sample with an antibody that specifically recognizes, or specifically binds to, the immune effector molecule (such as IFN-γ); and detecting the complex formed between the antibody and the immune effector molecule (such as IFN-γ).

The level of an immune effector molecule (such as IFN-γ) can be determined based on mRNA level. In one embodiment, the mRNA level of an immune effector molecule (such as IFN-γ) can be determined by a method comprising contacting the biological sample with a polynucleotide probe that comprises a nucleic acid sequence that specifically binds to, or hybridizes under stringent conditions with, the mRNA of an immune effector molecule (such as IFN-γ); and detecting the complex formed between the polynucleotide probe and the mRNA of an immune effector molecule (such as IFN-γ).

As used herein, “stringent” conditions for hybridization refers to conditions wherein hybridization is typically carried out overnight at 20-25° C. below the melting temperature (Tm) of the DNA hybrid in 6×SSPE, 5×Denhardt's solution, 0.1% SDS, 0.1 mg/ml denatured DNA. The melting temperature, Tm, is described by the following formula (Beltz et al., 1983):

Tm=81.5 C+16.6 Log [Na+]+0.41(% G+C)−0.61(% formamide)−600/length of duplex in base pairs.

Washes are carried out as follows:

(1) Twice at room temperature for 15 minutes in 1×SSPE, 0.1% SDS (low stringency wash).

(2) Once at Tm-20 C for 15 minutes in 0.2×SSPE, 0.1% SDS (moderate stringency wash).

In one embodiment, the mRNA level of an immune effector molecule (such as IFN-γ) can be determined by polymerase chain reaction methods. Polymerase chain reaction (PCR) is a process for amplifying one or more target nucleic acid sequences present in a nucleic acid sample using primers and agents for polymerization and then detecting the amplified sequence. The extension product of one primer when hybridized to the other becomes a template for the production of the desired specific nucleic acid sequence, and vice versa, and the process is repeated as often as is necessary to produce the desired amount of the sequence. The skilled artisan, to detect the presence of a desired sequence (U.S. Pat. No. 4,683,195), routinely uses polymerase chain reaction.

A specific example of PCR that is routinely performed by the skilled artisan to detect desired sequences is reverse transcript PCR (RT-PCR; Saiki et al., Science, 1985, 230:1350; Scharf et al., Science, 1986, 233:1076). RT-PCR involves isolating total RNA from biological fluid, denaturing the RNA in the presence of primers that recognize the desired nucleic acid sequence, using the primers to generate a cDNA copy of the RNA by reverse transcription, amplifying the cDNA by PCR using specific primers, and detecting the amplified cDNA by electrophoresis or other methods known to the skilled artisan.

Samples and/or binding agents specific for the immune effector molecule of interest (such as IFN-γ) may be arrayed on a solid support, or multiple supports can be utilized, for multiplex detection or analysis. “Arraying” refers to the act of organizing or arranging members of a library (e.g., an array of different samples or an array of devices that target the same target molecules or different target molecules), or other collection, into a logical or physical array. Thus, an “array” refers to a physical or logical arrangement of, e.g., biological samples. A physical array can be any “spatial format” or “physically gridded format” in which physical manifestations of corresponding library members are arranged in an ordered manner, lending itself to combinatorial screening. For example, samples corresponding to individual or pooled members of a sample library can be arranged in a series of numbered rows and columns, e.g., on a multi-well plate. Similarly, binding agents can be plated or otherwise deposited in microtitered, e.g., 96-well, 384-well, or 1536-well plates (or trays). Optionally, samples and/or binding agents specific for the immune effector molecule of interest (such as IFN-γ) may be immobilized on the solid support.

In one embodiment of an assay for determining the level or concentration of the immune effector molecule of interest (such as IFN-γ), an unlabeled antibody is immobilized on a solid substrate and the sample to be tested for the immune effectors brought into contact with the bound molecule. After a suitable period of incubation, for a period of time sufficient to allow formation of an antibody-immune effector molecule complex, a second antibody specific to the antibody-immune effector molecule complex, labeled with a reporter molecule capable of producing a detectable signal, is then added and incubated, allowing time sufficient for the formation of another complex of antibody-immune effector molecule complex-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of immune effector molecule. This generalized technique is well known to those skilled in the art as would be any of a number of variations.

In certain embodiments, a first antibody having specificity for the instant immune effectors is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, spheres, discs of microplates, or any other surface suitable for conducting an immunoassay. An aliquot of the sample to be tested is then added to the solid phase complex and incubated for a period of time sufficient (e.g. 2-120 minutes or where more convenient, overnight) and under suitable conditions (e.g. for about 20° C. to about 40° C.) to allow binding of any subunit present in the antibody. Following the incubation period, the antibody subunit solid phase is washed and dried and incubated with a second antibody specific for a portion of the immune effector molecule. The second antibody is linked to a reporter molecule, which is used to indicate the binding of the second antibody to the hapten.

The term “reporter molecule,” as used herein, refers to a molecule which, by its chemical nature, provides an analytically identifiable signal that allows the detection of antigen-bound antibody. Detection may be either qualitative or quantitative. The most commonly used reporter molecules in this type of assay are either enzymes, fluorophores or radionuclide containing molecules (i.e., radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable colour change. Examples of suitable enzymes include alkaline phosphatase and peroxidase. It is also possible to employ fluorogenic substrates, which yield a fluorescent product rather than the chromogenic substrates noted above. In certain embodiments, the enzyme-labeled antibody is added to the first antibody-immune effector molecule complex, allowed to bind, and then the excess reagent is washed away. A solution containing the appropriate substrate is then added to the complex of antibody-immune effector molecule-antibody. The substrate will react with the enzyme linked to the second antibody, giving a qualitative visual signal, which may be further quantitated, usually spectrophotometrically, to give an indication of the amount of immune effector molecule which was present in the sample. Again, the present invention extends to a substantially simultaneous assay.

In certain embodiments, fluorescent compounds, such as fluorescein and rhodamine, may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic colour visually detectable with a light microscope. The fluorescent labeled antibody is allowed to bind to the first antibody-antigen complex. After washing off the unbound reagent, the remaining tertiary complex is then exposed to the light of the appropriate wavelength; the fluorescence observed indicates the presence of the antigen of interest. Immunofluorescene and EIA techniques are both very well established in the art and are particularly preferred for the present method. However, other reporter molecules, such as radioisotope, chemiluminescent or bioluminescent molecules, may also be employed.

The present invention also contemplates genetic assays such as involving PCR analysis to detect RNA expression products of a genetic sequence encoding an immune effector. In one embodiment, PCR is conducted using pairs of primers, one or both of which are generally labeled with the same or a different reporter molecule capable of giving a distinguishable signal. The use of fluorophores is particularly useful in the practice of the present invention. Suitable labels can also include luminescence and phosphorescence as well as infrared dyes. These dyes or fluorophores may also be used as reporter molecules for antibodies.

The predetermined reference value can be readily established by skilled healthcare practitioners. For diagnosis of persistent Chlamydial infection, the predetermined reference value can be established, for example, by contacting peptide antigen molecules of the present invention with control biological samples (e.g. whole blood, plasma, serum, and blood cells) obtained from subjects who do not have persistent Chlamydial infection, and determining the level of an immune effector molecule of interest (such as IFN-γ) in the control samples. Usually, the predetermined reference value is the cut-off value that distinguishes patients with persistent Chlamydial infection from subjects without persistent Chlamydial infection. The presence or elevation of the level of the immune effector molecule (e.g., IFN-γ) in a subject's sample, as compared to the predetermined reference value, is diagnostic of persistent Chlamydial infection.

Preferably, the predetermined reference value is capable of distinguishing subjects with persistent Chlamydial infection from subjects with acute Chlamydial infection. Preferably, the control subjects do not have infection (e.g., bacterial, viral, fungal and other microbial infection), inflammation, or auto-immune diseases. For instance, the control subjects do not have M. tuberculosis or H. pylori infection. Further, the predetermined reference value is preferably provided by using the same type of biological sample and the same assay technique as is used for measurement of the subject's level (e.g., IFN-γ level), to avoid any error in standardization.

The present invention can also be used to diagnose diseases related to persistent Chlamydial infection, including diseases arising from, induced by, or associated with persistent Chlamydial infection (including Chlamydia trachomatis and Chlamydia pneumoniae) infection. Such diseases include, but are not limited to, Chlamydia-induced ReA, undifferentiated Spondyloarthritis (uSpA), trachoma, female infertility, coronary artery disease, asthma, and cervical cancer.

Further, for subjects who are diagnosed with persistent Chlamydial infection and/or diseases arising from persistent Chlamydial infection, the present invention can also be used to determine the scope or severity of persistent Chlamydial infection and/or diseases arising from persistent Chlamydial infection. Generally, a higher level of IFN-γ in response to stimulation by peptide antigen molecules indicates that the subject has more severe persistent Chlamydial infection and/or diseases arising from persistent Chlamydial infection.

In addition, the present invention can be used to determine the progression of persistent Chlamydial infection and/or diseases arising from persistent Chlamydial infection by determining IFN-γ levels in biological samples obtained at different time points. For instance, a progressive elevation of IFN-γ levels overtime indicates worsening of the persistent Chlamydial infection and/or diseases arising from persistent Chlamydial infection.

### Diagnostic Kits

A second aspect of the present invention provides kits for diagnosing persistent Chlamydial infection. In an embodiment, the kit comprises peptide antigen molecules derived from a Chlamydial HSP-60 protein that is expressed during persistent infection. In a specific embodiment, the kit comprises peptide antigen molecules that are fragments of, or otherwise derived from, Chlamydia proteins expressed or whose expression is upregulated during persistent infection.

In certain embodiments, the kit comprises peptide antigen molecules that are fragments, or derived from, a Chlamydia protein selected from SEQ ID NOs: 1-33. In certain embodiments, the kit comprises peptide antigen molecules that are fragments, or derived from, Chlamydia proteins including Chlamydia trachomatis (Ct) 604-encoded-protein (SEQ ID NO:1), Chlamydia trachomatis (Ct) 110-encoded protein (SEQ ID NO:2), Chlamydia trachomatis (Ct) 413-encoded protein (SEQ ID NO:24), Chlamydia trachomatis (Ct) 600-encoded protein (SEQ ID NO:33), and Chlamydia trachomatis (Ct) 849-encoded protein (SEQ ID NO:6). In certain embodiments, the kit comprises a peptide antigen comprising any of SEQ ID NOs: 34-37.

In certain specific embodiments, the kit further comprises an application zone for receiving a biological sample (such as a blood sample); a labeling zone containing a binding agent that binds to an immune effector molecule (e.g., IFN-γ) or mRNA in the sample; and a detection zone where an immune effector molecule (e.g., IFN-γ)-bound binding agent is retained to give a signal, wherein the signal given for a sample of a subject with the level of the immune effector molecule (e.g., IFN-γ) greater than a control level is different from the signal given for a sample of a subject with the level of the immune effector molecule (e.g., IFN-γ) lower than a control level.

In one embodiment, the kit comprises a polynucleotide probe that comprises a nucleic acid sequence that specifically binds to, or hybridizes under highly stringent conditions, an mRNA of an immune effector molecule (e.g., IFN-γ) of interest; and a primer set that amplifies the mRNA of an immune effector molecule (e.g., IFN-γ) of interest.

In another specific embodiment, the kit comprises peptide antigen molecules that are fragments of, or otherwise derived from, Chlamydia pneumoniae (Cpn) HSP-60 proteins expressed during persistent infection.

Optionally, the kit may include any material useful for performing the present diagnostic method as described above. For instance, the kit may further comprise agents that are useful for detection or visualization of antigen-induced immune responses (e.g., IFN-γ responses) in biological samples. Such agents include antibodies that recognize IFN-γ or reporter molecules that provide identifiable signals for analysis of IFN-γ levels. In addition, the kit may further comprise agents that preserve or maintain peptide antigen molecules.

The kit may also comprise, e.g., a buffering agent, a preservative, or a protein stabilizing agent. The kit may also comprise components necessary for detecting the detectable agent (e.g., an enzyme or a substrate). The kit may also contain a control sample or a series of control samples which can be assayed and compared to the test sample contained. Each component of the kit is usually enclosed within an individual container and all of the various containers are within a single package along with instructions.

The methods of the invention can be carried out using a diagnostic kit for qualitatively or quantitatively detecting IFN-γ in a sample such as blood. By way of example, the kit can contain binding agents (e.g., antibodies) specific for IFN-γ, antibodies against the antibodies labeled with an enzyme; and a substrate for the enzyme. The kit can also contain a solid support such as microtiter multi-well plates, standards, assay diluent, wash buffer, adhesive plate covers, and/or instructions for carrying out a method of the invention using the kit. In one embodiment, the kit includes one or more protease inhibitors (e.g., a protease inhibitor cocktail) to be applied to the biological sample to be assayed (such as blood).

The agent(s) can be packaged with a container for collecting the biological fluid from a patient. When the antibodies or binding partner are used in the kits in the form of conjugates in which a label is attached, such as a radioactive metal ion or a moiety, the components of such conjugates can be supplied either in fully conjugated form, in the form of intermediates or as separate moieties to be conjugated by the user of the kit.

### Generation of Peptide Antigens

A third aspect of the invention provides methods for generating peptide antigens that can be used to diagnose persistent Chlamydial infection and/or diseases arising from persistent Chlamydial infection.

In an embodiment, the method comprises:

a) providing a candidate peptide antigen derived from a Chlamydial protein expressed during persistent infection;

b) contacting the candidate peptide antigen with a sample comprising host cells having persistent Chlamydial infection, wherein the host cells are producing IFN-γ;

c) measuring a level of IFN-γ in the sample; and

d) selecting the candidate peptide antigen if the candidate peptide antigen increases the level of IFN-γ in the sample.

In one embodiment, the candidate peptide antigen is a fragment of, or derived from a Chlamydial protein selected from SEQ ID NOs: 1-33.

In an embodiment, a library of candidate peptide antigens is generated from Chlamydial HSP-60 protein expressed during persistent infection. Such candidate peptide antigens can be the full length, or antigenic fragments of, Chlamydial HSP-60 protein. In preferred embodiments, sequence analysis of the Chlamydial HSP-60 protein expressed or up-regulated during persistent infection as compared to that of protein expressed or up-regulated during acute infection is performed. Preferably, the candidate peptide antigen does not comprise sequences that are identical or substantially similar to the HSP-60 protein (or fragments thereof) expressed during acute infection.

Cells can be persistently infected with Chlamydiae in vitro or in vivo. For instance, biological samples such as blood samples (e.g., whole blood, plasma, serum, and blood cells) obtained from patients with persistent Chlamydial infection can be used. Alternatively, cells can be persistently infected with Chlamydiae in vitro, such as via incubation with Chlamydial peptides or proteins expressed during persistent infection.

Preferably, candidate peptide antigens are further screened so that they do not elicit immune responses (e.g., IFN-γ responses) in cells with acute Chlamydial infection. Alternatively, candidate peptide antigens are screened so that they elicit different levels of immune responses in cells with persistent Chlamydial infection, as compared to cells with acute Chlamydial infection. In another embodiment, candidate peptide antigens are screened so that they do not elicit immune responses (e.g., IFN-γ responses) in biological samples obtained from subjects who have non-Chlamydial infection (e.g., bacterial, viral, fungal and other microbial infection), inflammation, or auto-immune diseases. Alternatively, candidate peptide antigens are screened so that they elicit different levels of immune responses in samples with persistent Chlamydial infection, as compared to samples obtained from subjects who have non-Chlamydial infection (e.g., bacterial, viral, fungal and other microbial infection), inflammation, or auto-immune diseases.

### Treatment of Persistent Chlamydial Infection

A fourth aspect of the present invention provides treatment methods for persistent Chlamydial infection and/or related diseases. In an embodiment, the method comprises administering to a subject in need of such treatment an antibiotic agent. Preferably, combination antibiotic therapy is used. In a specific embodiment, the antibiotic agent is selected from azithromycin, rifampin, doxycycline, or any combination thereof.

The present inventors have discovered that persistent forms of chlamydiae are susceptible to antimicrobial therapy. Further, results of a 9-month open-label clinical trial demonstrate that the combination of doxycycline and rifampin more effectively alleviated symptoms of Chlamydia-induced ReA than doxycycline monotherapy. In a double-blind placebo-controlled clinical trial, treatment effects of a 6-month course of the combination antibiotics (either doxycycline and rifampin or azithromycin and rifampin) were superior to placebos. Combination antibiotic therapy not only alleviated symptoms of chronic Chlamydia-induced ReA, but also cleared Chlamydial infection.

### EXAMPLES

Following are examples that illustrate procedures for practicing the invention. These examples should not be construed as limiting. All percentages are by weight and all solvent mixture proportions are by volume unless otherwise noted.

### Example 1

### Generation of Peptide Antigens for Diagnosis of Persistent Chlamydia Trachomatis Infection

This Example illustrates methods for generating peptide antigens useful for diagnosing persistent Chlamydia trachomatis infection. In an embodiment, peptide antigens are derived from the HSP-60 homolog encoded by Ct604, which is significantly up-regulated during persistent infection both in vivo and in vitro. Preferably, the peptides are of 8 to 20 amino acids in length. Preferably, the peptides are not derived from regions of the Ct604-encoded homolog that have similar sequences to that of the Ct110-encoded homolog (SEQ ID NO:2). Regions of local similarity between sequences can be determined by conventional techniques such as the basic local alignment search tool (BLAST). During the course of designing candidate peptide antigens, one need not consider whether the Ct604-encoded HSP-60 homolog shares any sequence similarity to that of the Ct755-encoded homolog, which is essentially unexpressed during persistent infection.

Specifically, it is discovered by the present inventors that Ct604 encodes an HSP-60 homolog that shares about 25% sequence identity and about 45% similarity that of the Ct110-encoded homolog. For example, amino acid residues 112-170 of the Ct604-encoded homolog share significant sequence identity to amino acid residues 114-172 of the Ct110-encoded homolog. In addition, acid residues 474-497 of the Ct604-encoded homolog share significant sequence identity to amino acid residues of 478-501 and 287-308 of the Ct110-encoded homolog. Thus, the peptide antigens are, preferably, not derived from amino acid residues 112-170 and 474-497 of the Ct604-encoded homolog.

To select peptide antigens useful for diagnosis of persistent Chlamydial infection, blood samples isolated from patients with persistent Chlamydial infection are incubated with candidate peptide antigens for 16 to 24 hours. In addition, blood samples isolated from individuals without persistent Chlamydial infection (control) are incubated with candidate peptide antigens under the same conditions. The levels of IFN-γ are measured using ELISA. Peptide antigens that induce an elevated level of IFN-γ in the patient samples, as compared to that of the controls samples, are selected. To avoid false positive IFN-γ responses, the patients are screened so that they do not have infection such as undiagnosed latent M tuberculosis infections. For instance, patients with latent M tuberculosis infections can be detected using tuberculin skin test.

### Example 2

### Determination of the Cut-Off Value

This Example illustrates a preferred embodiment for determining the cut-off value indicative of persistent Chlamydial infection. Specifically, whole blood samples collected from individuals are subject to the PCR assay that identifies the presence of C. trachomatis DNA (such as C. trachomatis omp1 and 16S rRNA). The sample with detectable level of C. trachomatis DNA is considered as PCR-positive, which indicates that the individual has persistent Chlamydial infection. The sample without detectable level of C. trachomatis DNA is considered as PCR-negative, which indicates that the individual does not have persistent Chlamydial infection. The blood samples are incubated with peptide antigens of the present invention, and IFN-γ levels are determined. The cut-off value is determined based on the IFN-γ level that distinguishes the PCR-positive v. PCR-negative samples.

In a further embodiment, the correlation between the level of IFN-γ response and the degree of persistent Chlamydial infection can be determined. Specifically, synovial tissue samples of patients with persistent Chlamydia infection (PCR-positive in synovial tissue) are subject to qtPCR for determining the level of Chlamydial DNA. In addition, whole blood samples of the patients are incubated with peptide antigen molecules of the present invention, and IFN-γ levels in the blood samples are determined. The level of Chlamydial DNA in the synovial tissue and the IFN-γ level in the blood sample is analyzed using Kappa and Pearson's correlation analysis to assess the relationship between the IFN-γ responses and the level of Chlamydia DNA in the synovial tissue samples.

### Example 3

### Absence of Chlamydia DNA in Urine Samples of Patients with Persistent Chlamydial Infection

This Example reveals that urine samples of patients with persistent Chlamydial infection contain no detectable level of Chlamydia DNA. Briefly, urine samples of patients with Chlamydia-induced ReA are subject to PCR analysis and no detectable Chlamydia DNA is determined. This reveals that during persistent infection such as Chlamydia-induced ReA, arthritogenic Chlamydial serovars completely vacate the genital area, which is the initial site of infection, and migrate into distant sites such as ocular tissues, synovial tissues and blood.

### Example 4

### Use of Interferon-Gamma-Based Assay for Diagnostic of Persistent Chlamydia Infection

A blood sample (approximately 15 mL's of blood) is obtained from a patient who is known to have Chlamydia-induced reactive arthritis. This patient also previously had a synovial biopsy of the knee and PCR analysis shows the presence of persistent Chlamydia trachomatis infection. From this blood sample, monocytes (MNCs) are prepared and plated on a six well plate (900,000 per well). Peptide antigens (100 uM) are dissolved in phosphate buffered solution (PBS) and incubated for 30 min at 37° C. After incubation, MNCs are washed and then RPMI40 medium is added.

In each of the six wells, monocytes are incubated with the following: PBS (without any peptide antigen), CT600 antigen, C849 antigen, CT 413-2 antigen, CT 413-3 antigen, and a scramble peptide antigen at a concentration of 100 uM, respectively.

The CT600, CT849, CT413-2, and CT413-3 are four different Chlamydia trachomatis peptide antigens that are specific for patients with persistent Chlamydial infection. The “scrambled” peptide is a random amino acid sequence utilized as one of the controls.

The amino acid sequences of the CT600, CT849, CT413-2, and CT413-3 antigens are as follows:

CT600 (predicted to be an outer membrane protein):

| | |
|---|---|
| PKATLYIEGHTDERGAAAYN; | (SEQ ID NO: 34) |

CT413-2 (predicted to be an extracellular/secreted protein):

| | |
|---|---|
| EYIVSGNASFTKFTNIPT; | (SEQ ID NO: 35) |

CT413-3 (predicted to be an extracellular/secreted protein):

| | |
|---|---|
| QLYLGPFWTLYGNYTIDVG; | (SEQ ID NO: 36) |

CT849 (predicted to be an inner membrane protein)

| | |
|---|---|
| SFIKTLNSVGSTVNQLNKPLS. | (SEQ ID NO: 37) |

The reaction is stopped after 3 hours and RNA is extracted using TRIZOL®. DNA is eliminated by DNase treatment, and reverse transcription is performed with 2.5 ug of RNA. Real-Time analysis targeting the 18S rRNA and Interferon-gamma is performed. The results are normalized with 18S and compared with the scrambled peptide.

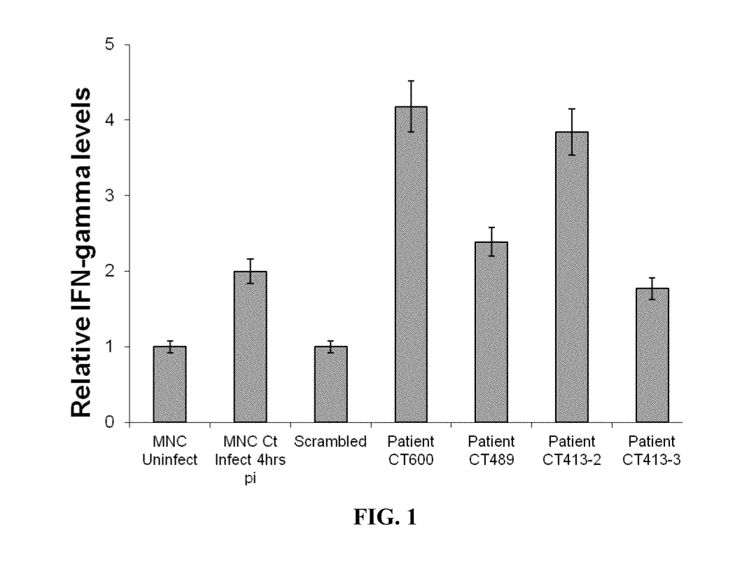

As shown in FIG. 1, monocytes incubated with CT600, CT849, CT413-2, and CT413-3 antigens have significantly elevated levels of interferon-gamma (a 2- to 4-fold increase in interferon-gamma level), when compared to monocytes incubated with PBS only or scrambled peptides. The control wells (PBS- and scrambled peptide-incubated wells) produce no interferon-gamma responses. Human monocytes uninfected or infected with Chlamydia also serve as controls. Human monocytes infected with Chlamydia produce a 2-fold increase in interferon-gamma level, when compared to monocytes incubated with PBS only or scrambled peptides.

The results show that blood samples of patients with Chlamydia-induced reactive arthritis and chlamydial persistence, when exposed to chlamydial peptides that are unique and specific to persistent Chlamydial infections, produce an elevated interferon-gamma response. This Example shows that interferon-gamma based assay of the current invention can be used for diagnosing persistent Chlamydial infections.

### Example 5

### Treatment Effects of Combination Antibiotics on Persistent Chlamydial Infection

In a randomized, 9-month prospective, blinded, placebo-controlled study illustrated below, combination antibiotic therapy resulted in improvement in clinical outcomes in patients with chronic Chlamydia-induced ReA. Subjects are considered as having chronic Chlamydia-induced ReA if they meet the following criteria: 1) the subjects meet a modified European Spondyloarthropathy Study Group Criteria (ESSG) with a minimal disease duration of six months (i.e., chronic patients); 2) the subjects have Ct or Cpn DNA in synovial tissue or PBMC based on PCR analysis.

PCR analysis of the presence of Ct or Cpn DNA in synovial tissue of patients who already meet the clinical criteria for ReA or undifferentiated spondyloarthritis represents the state-of-the-art means to accurately and specifically identify Chlamydial infection as the etiology. Thus, patients who met the above criteria did not have post-enteric ReA or other types of spondyloarthritis (SpA). Of 42 subjects, 27 had Ct or Cpn DNA in PBMC and 15 had Ct or Cpn DNA in synovial tissue.

Patients with Chlamydia-induced ReA were randomized in a blinded fashion and treated with combination of azithromycin and rifampin or doxycycline and rifampin. Rifampin is a bactericidal antibiotic agent that blocks the production of chlamydial HSPs. All subjects were treated in a blinded fashion for six months and followed for three more months after cessation of their therapy (to ensure symptoms did not “rebound” after discontinuing treatment).

The results demonstrated that a 6-month course of combination antibiotics resulted in a significantly higher response rate in subjects with chronic Chlamydia-induced ReA, as compared to placebos. Specifically, at Month 6 (primary endpoint), 17/27 subjects (63%) randomized to combination antibiotics were responders compared to 3/15 (20%) who received placebos (P-value=0.01). Subjects randomized to combination antibiotics also showed significant improvement in the modified swollen joint count, tender joint count, physician global assessment (P-values 0.0007, 0.002, and 0.0009, respectively), and a trend with the erythrocyte sedimentation rate (P-value=0.07) compared to subjects who received placebos.

A post-hoc analysis was performed to further determine the effects of combination antibiotics on Chlamydia-induced ReA. Responders to combination antibiotic therapy are defined as those subjects with at least 20% improvement in at least 4 of 6 variables without worsening in any one variable. These 6 variables include modified swollen joint count (SJC), tender joint count (TJC), average duration of morning stiffness in low back per day over the past week, current low back pain visual analog scale (VAS), current peripheral joint pain VAS, and global VAS. 50% and 70% responders refer to subjects with at least 50% and 70% improvement in at least 4 of 6 variables without worsening in any one variable, respectively. Of the 27 subjects randomized to combination antibiotics, 11/27 (41%) were 50% responders and 7/27 (26%) were 70% responders. In comparison, only 1/15 (7%) of the subjects who received placebos were 70% responders.

Further, while acute ReA may remit spontaneously, it is unlikely that symptoms of chronic ReA would improve or resolve spontaneously. Here, 22% (6/27) of the subjects randomized to combination antibiotics had ReA symptoms completely resolved, whereas no placebo-treated subjects achieved remission. Further, all of the subjects maintained remission at least for three month after the completion of antibiotic therapy. PCR results also showed absence of Chlamydia DNA in PBMC and synovial tissue samples in subjects who completed 6 months of antibiotic therapy (P-value=0.03).

In addition, 67% of the subjects randomized to azithromycin and rifampin were responders, whereas 58% randomized to doxycycline and rifampin were responders. Interestingly, 5/6 of all patients who achieved remission were randomized to the combination of azithromycin and rifampin, indicating that 5/15 (33%) of the subjects on azithromycin and rifampin achieved remission.

### Example 6

### Optimization of Combination Antibiotic Therapy for Persistent Chlamydial Infection

This Example investigates three primary endpoints and eight secondary endpoints of the combination antibiotics therapy for persistent Chlamydial infection. The primary endpoints include: 1) a comparison of the number of responders randomized to azithromycin (low-dose and high-dose) and rifampin versus doxycycline and rifampin after six months of active therapy; 2) a comparison of the number of responders on the three different combination antibiotic treatment strategies—azithromycin (low-dose) and rifampin; azithromycin (high-dose) and rifampin; doxycycline and rifampin, as compared to placebo at Month 3; and 3) a comparison of clearance rate of persistent Chlamydial infection in synovial tissue as a result of 3 months versus 6 months of combination antibiotics (based on synovial tissue PCR analysis after 3 versus 6 months of blinded therapy).

This Example also investigates secondary endpoints, including: 1) a comparison of the number of 50% and 70% responders of the three different combination antibiotic treatment strategies versus placebo at Month 3; 2) a comparison of the number of 50% and 70% responders randomized to azithromycin (low-dose and high-dose) and rifampin versus doxycycline & rifampin after six months of therapy: 3) a comparison of the number of responders randomized to azithromycin (high dose) and rifampin versus azithromycin (low dose) and rifampin versus doxycycline and rifampin; 4) evaluation of the clearance rate of persistent Chlamydial infection based on PCR analysis of PBMC samples at 1, 3, 6, 9, and 12 months of treatment in groups 1, 2, and 3; 5) a comparison of patients with at least 20% worsening of persistent Chlamydial infection, three versus six months after the completion of the combination antibiotic therapy; 6) a comparison of acute phase reactants (Erythrocyte Sedimentation Rate [ESR] and highly sensitive C-reactive protein [hsCRP]) in treatment groups 1, 2, and 3 at months 1, 3, 6, 9, and 12; 7) evaluation of cytokine levels using ELISA at screening, baseline, months 3, 6, 9, and 12; and 8) evaluation of the number of patients in remission after six months of therapy. In this Example, remission means that disease-related symptoms were “100% resolved”.

Patients are selected from participants with either uSpA or suspected Chlamydia-induced ReA. Subjects are considered as suffering from uSpA if he/she fulfills the criteria for SpA as defined by the ESSG, without evidence suggesting a more specific disorder, such as ankylosing spondylitis (AS), inflammatory bowel disease (IBD), psoriasis, or a preceding bacterial trigger. Patients with a preceding enteric infection as the trigger are excluded in order to eliminate post-dysentery ReA patients, according to the inclusion and exclusion criteria illustrated below. In addition, all subjects have active synovitis (at least one tender or swollen joint) in order to be randomized. This eliminates subjects with only axial disease because these subjects are likely to have early “pre-radiographic” AS rather than ReA.

Specifically, inclusion criteria are:

1. disease duration of at least 6 months;

2. fulfillment of a modified European Spondyloarthropathy Study Group Criteria;

2a) synovitis; and

2b) one or more of the following:

i) inflammatory spinal pain; ii) positive family history; iii) urethritis or cervicitis within 1 month before arthritis; iv) buttock pain; v) enthesopathy; and vi) radiographic sacroiliitis (at least grade II unilateral sacroiliitis).

Exclusion criteria include:

1. inflammatory arthritis other than ReA (other than gout);

2. psoriasis;

3. history inflammatory bowel disease;

4. sensitivity or history of allergic reaction to rifampin, doxycycline, or azithromycin;

5. taking concurrent medications that may interact with any of rifampin, doxycycline, or azithromycin, specifically rifampin;

6. liver transaminases greater than or equal to 2 times normal;

7. significant abnormalities in the CBC;

8. pregnancy;

9. prior prolonged exposure to antibiotics (>2 weeks) as a potential treatment for ReA; and

10. ankylosing spondylitis, psoriatic arthritis, spondyloarthritis patients with axial disease alone.

Blood samples collected from the subjects are subject to PCR analysis to determine the presence of C. trachomatis DNA. Subjects with knee synovitis are subject to synovial tissue biopsy to determine the presence of C. trachomatis or C. pneumoniae in synovial tissue using PCR. Only those study subjects who meet the diagnostic criteria for uSpA or suspected Chlamydia-induced ReA and whose PBMC samples are PCR-positive for Ct or whose synovial tissue/fluid samples are PCR-positive for Ct or Cpn are randomized to treatment. In this Example, subjects who are PCR-positive for Cpn in their PBMC are not considered as patients because a significant percentage of the population (5-40%) are PCR-positive for Cpn in their PBMCs. AS a result, PCR-positive for Cpn is not considered as diagnostic of Chlamydia-induced ReA, even if the subjects meet other criteria (since postenteric ReA and other types of SpA can be difficult to differentiate from post-chlamydial ReA).

To determine the presence of Chlamydial DNA in synovial tissue, blood or other biological samples, PCR analysis using primers derived from C. trachomatis and/or C. pneumoniae DNA is performed using methods known in the art. Primers for determining the presence of C. trachomatis DNA are derived from Ct omp1 and/or 16S rRNA genes. Primers for determining the presence of C. pneumoniae DNA are derived from homologous genes. Preferably, Ct-directed primers do not amplify sequences of Cpn, and vice-versa. If Ct or Cpn DNA is detected, the sample is considered as PCR-positive. Extreme care should be taken to avoid contamination of PCR-related materials.

105 patients are double-randomized (1:1:1) to receive azithromycin (high-dose) & rifampin or azithromycin (low-dose) & rifampin or doxycycline & rifampin for 6 months, and thereafter randomized (1:1) a second time to receive combination antibiotic therapy immediately or in 3 months (i.e., 3 months of placebo treatment followed by 6 months of combination antibiotics).

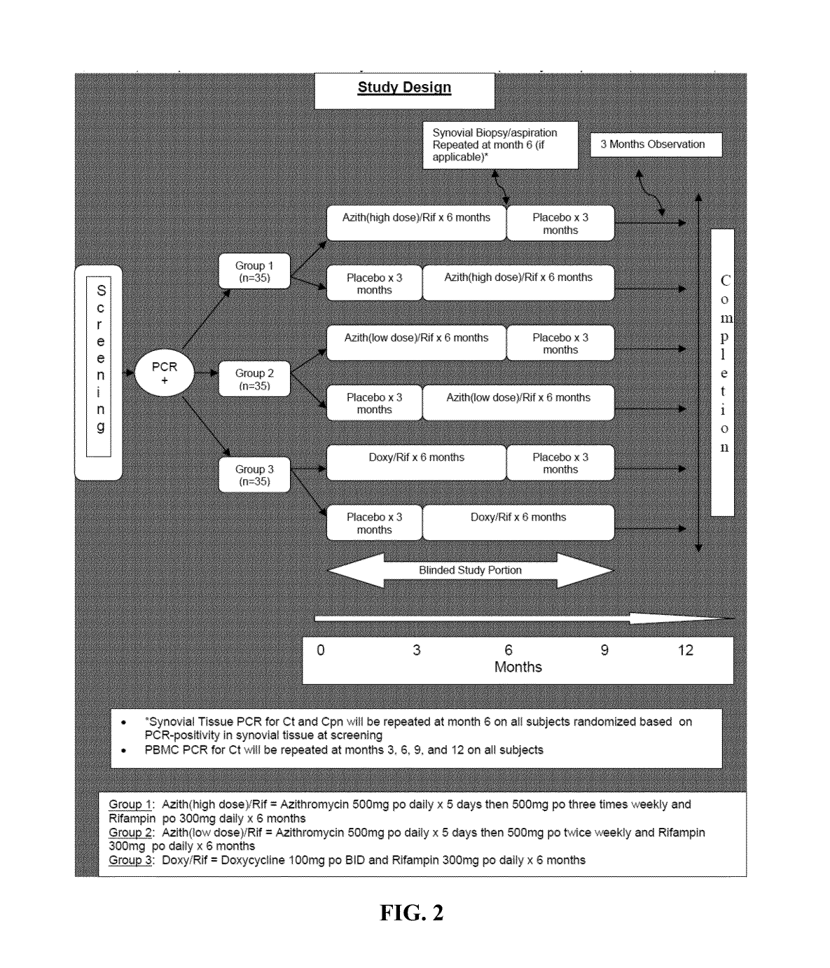

As shown in FIG. 2, the double-randomization is performed prior to the baseline visit. All patients are first randomized to receive azithromycin (high-dose)/rifampin, azithromycin (low-dose)/rifampin, or doxycycline/rifampin in a 1:1:1 fashion, and then immediately randomized again in a 1:1 fashion to receive either 3 months of placebo followed by 6 months of active therapy or 6 months of active therapy followed by 3 months of placebo. All patients receive blinded medication for a total of 9 months and are observed an additional 3 months after completion of the antibiotic treatment. This second randomization allows for comparison of different combination antibiotic strategies in a head-to-head fashion.

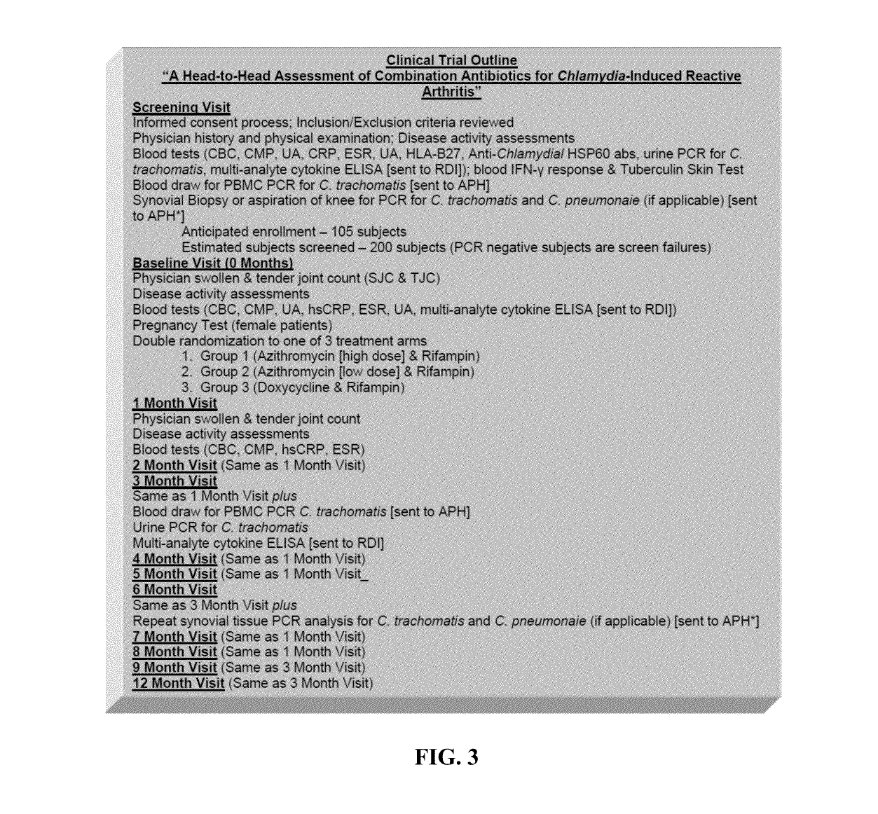

The patients who are randomized based on a PCR-positive synovial tissue analysis at baseline have a repeat synovial tissue biopsy from the same joint at Month 6. The patients randomized on PCR-positive PBMC results at baseline have a repeat PBMC PCR testing at Month 6 (along with other time-points as shown in FIG. 3). The patients who are randomized on PCR-positive synovial fluid results at baseline have a repeat aspiration from the same joint at Month 6; if their joint effusion has resolved at Month 6, a synovial tissue biopsy is performed for PCR analysis. (If the local IRB does not allow for synovial tissue biopsy at Month 6 in the patients whose effusions have resolved, PBMC PCR analysis is used instead). Analyzing all patients at Month 6 with clinical assessments and a repeat PCR analysis, allows for blinded comparison of 3 months versus 6 months of therapy in terms of clinical response and clearance rate of Chlamydial infection.

As outlined in FIG. 2, all patients receive active antibiotic therapy (i.e. treatment Group 1, 2 or 3) for 6 months and are blinded to receive combination antibiotic medications for 9 months. This novel double-randomization also includes three key assessments. The Month 3 visit serves as the placebo-controlled visit. At this visit, half of the patients receive active antibiotic therapy and the other half receive placebos. This allows a blinded, placebo-controlled comparison for each of the three combinations of antibiotics. At the Month 6 visit, half of the patients have completed all 6 months of active therapy and the other half have completed three months of active combination antibiotics, thereby allowing a blinded analysis of 6 versus 3 months of therapy by evaluating clearance of persistent Chlamydial infection by PCR analysis coupled with clinical responses. Finally, the Month 9 visit provides the blinded data necessary to determine the number of responders randomized to azithromycin (both dosing strategies) and rifampin versus doxycycline and rifampin after six months of active therapy.

In a double-blind, placebo-controlled, combination antibiotic trial (submitted to the 2010 American College of Rheumatology Convention), the present inventors have discovered that combination antibiotic therapy reduces cytokine levels of patients with persistent Chlamydial infection. Briefly, the levels of 42 cytokines in sera collected from patients who received 0, 6 and 9 months of randomized treatment were assessed using multi-analyte ELISA. Analysis of covariance (ANCOVA) was used to determine the treatment effects on cytokine values at a second time point after removing the variance account of the cytokine values at the first time point. Using a univariate analysis, the present inventors discovered that combination antibiotic therapy significantly reduces EGF, GM-CSF, IFN-γ, IL-1ra, TGF-α, VEGF, and IL-12p70 (p<0.05). Specifically, the combination antibiotic therapy most significantly reduces the levels of IL-12p70 (p<0.001). After Bonferroni adjustment for multiple test correction, changes in IL-12p70 levels as a result of antibiotic treatment remain significant (at both 6 months and 9 months).

IL-12p70 is required for optimal host IFN-γ T cell response against intracellular pathogens and endocervical IL-12 expression has previously been shown to decline with clearance of Chlamydia. The distinctive changes in serum levels of IL-12p70 following antibiotic treatment in patients with Chlamydia-induced ReA suggest changing host cytokine responses concurrent with clinical improvement.

The results show that 76% of the patients randomized to azithromycin and rifampin (Groups 1 and 2) are responders after 6 months of therapy and 58% of subjects randomized to doxycycline and rifampin are responders (alpha 5%, power 80%). In addition, 85% of the patients treated with azithromycin (high-dose)/rifampin (Group 1) and 67% of the patients treated with azithromycin (low-dose)/rifampin (Group 2) are responders. In addition, 58% of the patients who receive doxycycline/rifampin are responders, as compared to placebo (20%) (alpha 5%, power 80%).

To investigate Primary Endpoint 3), 38 study subjects are randomized based on a positive synovial tissue PCR analysis for Chlamydiae at screening (Subjects randomized at baseline on PCR-positive results from synovial fluid are excluded from these 38 patients). Synovial-based Chlamydial infection is cleared in 80% of the patients after 6 months of combination antibiotic therapy and in 50% of the patients after 3 months of combination antibiotic therapy. 30 patients must have a synovial biopsy at initial screening and at Month 6, which is 3 or 6 months after active antibiotic therapy depending on the randomization strategy) [alpha 5%, power 80%.] The patients randomized based on positive synovial-tissue PCR results for Chlamydiae are stratified at randomization to ensure equal number of patients in each of the three treatment groups.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

All patents, patent applications, provisional applications, and publications referred to or cited herein are incorporated by reference in their entirety, including all figures and tables, to the extent they are not inconsistent with the explicit teachings of this specification.

We claim:

1. A method of diagnosing persistent Chlamydial infection comprises:
a) obtaining a biological sample from a subject, wherein the biological sample contains immune cells capable of producing IFN-γ;
b) contacting the biological sample with a peptide antigen;
c) determining a level of IFN-γ in the biological sample; and
d) comparing the level of IFN-γ in the biological sample to a predetermined reference value;
wherein the peptide antigen is derived from Chlamydial heat shock protein-60 (HSP-60) expressed during persistent infection; and
wherein an elevated level of IFN-γ in the biological sample compared to the predetermined reference value indicates that the subject has persistent Chlamydial infection.

2. The method according to claim 1, wherein the biological sample is a blood sample.

3. The method according to claim 1, wherein the peptide antigen is derived from Chlamydial heat shock protein-60 (HSP-60) that is expressed during persistent infection, but is not expressed during acute infection.

4. The method according to claim 1, wherein the peptide antigen is derived from a Chlamydial heat shock protein-60 selected from the group consisting of Chlamydia trachomatis (Ct) protein encoded by Ct604, Chlamydia trachomatis (Ct) protein encoded by Ct110, Chlamydia trachomatis (Ct) protein encoded by Ct849, Chlamydia trachomatis (Ct) protein encoded by Ct413, and Chlamydia trachomatis (Ct) protein encoded by Ct600.

5. The method according to claim 1, wherein the peptide antigen is derived from a Chlamydial heat shock protein-60 selected from SEQ ID NOs:1-33.

6. The method according to claim 4, wherein the peptide antigen is derived from a Chlamydial heat shock protein-60 selected from the group consisting of SEQ ID NOs: 1, 2, 6, 24, and 33.

7. The method according to claim 1, wherein the peptide antigen is about 8 to 25 amino acids in length.

8. The method according to claim 1, wherein the peptide antigen comprises any of SEQ ID NOs: 34-37.

9. The method according to claim 1, wherein the level of IFN-γ in the biological sample is determined using enzyme-linked immunosorbant assay (ELISA), Western blot, immunoprecipitation, immunofluorescence, radioimmunoassay, immunocytochemistry, reverse transcription polymerase chain reaction (RT-PCR), enzyme-linked immunosorbent spot (ELISpot) assay, Northern blot, nucleic acid hybridization technique, fluorescent polarization (FO) technology, nucleic acid amplification technique, transcription mediated amplification (TMA), DNA strand displacement amplification (SDA), or a combination thereof.

10. The method according to claim 9, wherein the level of IFN-γ in the biological sample is determined using enzyme-linked immunosorbant assay (ELISA), Western blot, immunoprecipitation, immunofluorescence, radioimmunoassay, immunocytochemistry, enzyme-linked immunosorbent spot (ELISpot) assay, or a combination thereof.

11. The method according to claim 9, wherein the level of IFN-γ in the biological sample is determined using reverse transcription polymerase chain reaction (RT-PCR) and/or nucleic acid hybridization technique.

12. The method according to claim 1, wherein the level of IFN-γ protein expression is determined.

13. The method according to claim 12, wherein the level of IFN-γ in the biological sample is determined by contacting the biological sample with an antibody, aptamer, or binding partner that specifically binds to IFN-γ.

14. The method according to claim 1, wherein the level of IFN-γ mRNA is determined.

15. The method according to claim 14, wherein the level of IFN-γ in the biological sample is determined by contacting the biological sample with a polynucleotide probe that comprises a nucleic acid sequence that hybridizes under stringent conditions with IFN-γ mRNA; and detecting the complex formed between the polynucleotide probe and the IFN-γ mRNA.

16. The method according to claim 1, wherein the subject is suspected of having persistent Chlamydial infection.

17. The method according to claim 1, used to diagnose a disease arising from persistent Chlamydial infection.

18. The method according to claim 17, wherein the disease is selected from the group consisting of Chlamydia-induced reactive arthritis, undifferentiated Spondyloarthritis (uSpA), Chlamydia-induced trachoma, Chlamydia-induced female infertility, Chlamydia-induced coronary artery disease, Chlamydia-induced asthma, Chlamydia-induced cervical cancer.

19. A kit for diagnosing persistent Chlamydial infection, wherein the kit comprises
an antibody, apatamer, or binding agent that binds specifically to a peptide antigen molecule derived from a Chlamydial heat shock protein-60 selected from the group consisting of Chlamydia trachomatis (Ct) protein encoded by Ct604, Chlamydia trachomatis (Ct) protein encoded by Ct110, Chlamydia trachomatis (Ct) protein encoded by Ct849, Chlamydia trachomatis (Ct) protein encoded by Ct413, and Chlamydia trachomatis (Ct) protein encoded by Ct600;
an application zone for receiving a blood sample;
a labeling zone containing a binding agent that binds to IFN-γ or mRNA in the sample; and
a detection zone where an IFN-γ-bound binding agent is retained to give a signal, wherein the signal given for a sample of a subject with the IFN-γ level greater than a control level is different from the signal given for a sample of a subject with the IFN-γ level lower than a control level.

20. The kit according to claim 19, wherein the peptide antigen comprises any of SEQ ID NOs: 34-37.
